(19) United States
(12) Patent Application Publication
(10) Pub. No.: US 2025/0304892 A1
(43) Pub. Date: Oct. 2, 2025

(54) APPARATUS, SYSTEM, AND METHOD FOR FORMING A PERTURBABLE BONE MARROW MODEL WITHIN A THREE-DIMENSIONAL MICROPHYSIOLOGICAL SYSTEM

(71) Applicants: The Trustees of the University of Pennsylvania, Philadelphia, PA (US); THE CHILDREN'S HOSPITAL OF PHILADELPHIA, Philadelphia, PA (US)

(72) Inventors: Joseph Hai OVED, Philadelphia, PA (US); G. Scott WORTHEN, Philadelphia, PA (US); Andrei GEORGESCU, Philadelphia, PA (US); Dongeun HUH, Villanova, PA (US)

(21) Appl. No.: 18/948,800

(22) Filed: Nov. 15, 2024

Related U.S. Application Data
(63) Continuation of application No. PCT/US2023/067340, filed on May 23, 2023.
(60) Provisional application No. 63/365,150, filed on May 23, 2022.

(51) Int. Cl. C12M 3/00; C12M 1/00; C12N 5/071; C12N 5/0775; C12N 5/0789

(52) U.S. Cl. CPC C12M 21/08; C12M 23/34; C12M 29/00; C12N 5/0647; C12N 5/0668; C12N 5/069; C12N 2533/40; C12N 2533/54; C12N 2533/56; C12N 2533/72; C12N 2533/76; C12N 2533/90

(57) ABSTRACT

The present disclosure relates to a human bone marrow model. The present disclosure further relates to a microphysiological device comprising two or more side channels having endothelial cells therein and at least one central channel arranged therebetween, the at least one central channel having a cellularized scaffold formed therein, the cellularized scaffold of the at least one central channel including hematopoietic stem cells, mesenchymal stromal cells, and endothelial cells. In an embodiment, the device further comprises vasculature developed within and between the two or more side channels and the at least one central channel, wherein the vasculature developed within and between the two or more side channels and the at least one central channel includes anastomoses formed between vessels within the at least one central channel and an endothelium formed within the two or more side channels, the anastomoses permitting perfusion between the two or more side channels.

### CROSS REFERENCE TO RELATED APPLICATIONS

The present application is a continuation of PCT/US2023/067340, “Apparatus, System, And Method For Forming A Perturbable Bone Marrow Model Within A Three-Dimensional Microphysiological System,” filed May 23, 2023; which claims priority to and the benefit of U.S. patent application No. 63/365,150, “Apparatus, System, And Method For Forming A Perturbable Bone Marrow Model Within A Three-Dimensional Microphysiological System,” filed May 23, 2022. All foregoing applications are incorporated herein by reference in their entireties for any and all purposes.

### GOVERNMENT RIGHTS

This invention was made with government support under HL127720, TR002198 and TR001879 awarded by the National Institutes of Health and 1548571 awarded by the National Science Foundation. The government has certain rights in the invention.

### BACKGROUND

Certain organ-on-chip devices can include a micro-engineered biological cell-culture compartment in which tissue- and organ-level elements of human physiology can be recapitulated. The micro-engineered biological cell-culture compartment can allow physiological in vitro modeling of functional biological units of that organ system (e.g. insulin-secreting islet units to mimic a pancreas, air-liquid interfaces to mimic oxygen transport in the lung). Such models can enable evaluation of therapeutic interventions, diagnostic evaluations, and the like on live human tissue without requiring live human subjects.

Bone marrow plays certain roles in homeostatic mechanisms in the human body, including during tissue injury or turnover. To this end, self-renewable stem cells are capable of replenishing various types of specialized cells during tissue injury or turnover. Hematopoietic stem cells (HSCs) in particular can produce multipotent progenitor cells that give rise to different lineages of mature blood cells required for tissue oxygenation, hemostasis, and immunity. The highly orchestrated, lifelong process of hematopoiesis occurs in the perivascular regions of the bone marrow in which a majority of HSCs reside. This specialized microenvironment, referred to as the bone marrow niche, can play an essential role in hematopoiesis by regulating the activity of HSCs and hematopoietic progenitor cells.

Recent advances in genetic engineering and imaging technologies have greatly improved our understanding of the bone marrow niche. Native hematopoietic microenvironment can contain not only HSCs but also their progeny and various types of nonhematopoietic stromal cells organized into a complex multicellular network in the perivascular regions of the marrow. These cells produce a variety of membrane-bound and soluble niche factors that act in concert with biophysical cues provided by the extracellular environment to regulate the self-renewal and differentiation of HSCs. Elucidating how these intricate cellular and molecular interactions contribute to hematopoietic homeostasis and malignancies can be central to hematology and stem cell biology.

Despite some progress in this area, investigating human hematopoiesis remains a major challenge. Due to the inaccessibility of the human marrow, much of our understanding of hematopoiesis and the associated niche comes from mouse studies. With the growing recognition of significant species-specific differences in the hematopoietic system, however, increasing efforts are being made to complement animal studies with the use of human cell-based in vitro models. The primary focus of these studies thus far has been on creating in vitro analogs of the microenvironment adjacent to the endosteal bone surfaces, known as the endosteal niche, to emulate its role in HSC maintenance and differentiation. As increasing evidence shows close anatomical and functional association of HSCs with blood vessels, however, the focus of research has recently shifted towards elucidating the role of the vascular niche as a key regulator of hematopoiesis. While progress has been made in animal studies, in vitro investigation of the human-specific hematopoietic vascular niche represents a significant challenge. A major impediment to these types of studies has been the difficulty of recreating the complex, specialized microenvironment of the vasculature in the human marrow and its capacity to support HSC self-renewal and hematopoiesis.

Accordingly, there remains a need to create in vitro bone marrow niche analogs that mimic key physiological processes and allow for evaluation of the impact of injury and the like on the hematopoietic vascular niche of the human marrow.

### SUMMARY

The present disclosure relates a device, system, and methods of a bone marrow model. In particular, the present disclosure relates to a model of human bone marrow.

According to an embodiment, the present disclosure further relates to a microphysiological device, comprising two or more side channels, each of the two or more side channels having endothelial cells therein, and at least one central channel arranged therebetween, the at least one central channel having a cellularized scaffold formed therein, the cellularized scaffold of the at least one central channel including hematopoietic stem cells, mesenchymal stromal cells, and endothelial cells.

According to an embodiment, the present disclosure further relates to a microphysiological system for multi-organ modeling, comprising a first microphysiological device having two or more side channels and at least one central channel arranged therebetween, each of the two or more side channels having endothelial cells therein, the at least one central channel having a cellularized scaffold formed therein, the cellularized scaffold of the at least one central channel including hematopoietic stem cells, mesenchymal stromal cells, and endothelial cells, and wherein the first microphysiological device includes vasculature developed within and between the two or more side channels and the at least one central channel, a second microphysiological device having a plurality of chambers, the plurality of chambers including an apical chamber having epithelial cells therein, a central chamber having a cellularized scaffold formed therein, the cellularized scaffold of the central chamber including fibroblasts, and a basal chamber having endothelial cells therein, and a pump arranged in a fluidic circuit with the first microphysiological device and the second microphysiological device, an outflow of the first microphysiological device being an inflow to the second microphysiological device, an outflow of the second microphysiological device being an inflow to the first microphysiological device.

According to an embodiment, the present disclosure further relates to a method of preparing a microphysiological device comprising two or more side channels and at least one central channel arranged therebetween, comprising: a) contacting a scaffold of the at least one central channel with a plurality of hematopoietic stem cells, mesenchymal stromal cells, and endothelial cells, forming a cellularized scaffold in the at least one central channel, and b) contacting each of the two or more side channels with a plurality of endothelial cells.

The foregoing paragraphs have been provided by way of general introduction and are not intended to limit the scope of the following claims. The described embodiments, together with further advantages, will be best understood by reference to the following detailed description taken in conjunction with the accompanying drawings.

### BRIEF DESCRIPTION OF THE DRAWINGS

The file of this patent or application contains at least one drawing/photograph executed in color. Copies of this patent or patent application publication with color drawing(s)/photograph(s) will be provided by the Office upon request and payment of the necessary fee.

A more complete appreciation of the disclosure and many of the attendant advantages thereof will be readily obtained as the same becomes better understood by reference to the following detailed description when considered in connection with the accompanying drawings, wherein:

FIG. 1 is an illustration of a microphysiological device, according to an exemplary embodiment of the present disclosure;

FIG. 2A is a flow diagram of a method of a microphysiological device, according to an exemplary embodiment of the present disclosure;

FIG. 2B is an illustration of a flow diagram of a method of a microphysiological device, according to an exemplary embodiment of the present disclosure;

FIG. 3A is an illustration of a microphysiological device at a first time point after performing a method of the microphysiological device, according to an exemplary embodiment of the present disclosure;

FIG. 3B is an illustration of a microphysiological device at a second time point after performing a method of the microphysiological device, according to an exemplary embodiment of the present disclosure;

FIG. 3C is an illustration of a microphysiological device at a third time point after performing a method of the microphysiological device, according to an exemplary embodiment of the present disclosure;

FIG. 4 is an illustration of a microwell plate that includes at least one microphysiological device, according to an exemplary embodiment of the present disclosure;

FIG. 5 is an illustration of a lung-bone marrow multiorgan model, according to an exemplary embodiment of the present disclosure;

FIG. 6A is a flow diagram of a lung-bone marrow multiorgan model, according to an exemplary embodiment of the present disclosure;

FIG. 6B is an illustration of a lung-bone marrow multiorgan model, according to an exemplary embodiment of the present disclosure;

FIG. 7A is a graphical illustration of number of cells released from a first microphysiological device in response to infection of a second microphysiological device, according to an exemplary embodiment of the present disclosure;

FIG. 7B is a graphical illustration of a type of cells released from a first microphysiological device in response to infection of a second microphysiological device, according to an exemplary embodiment of the present disclosure; and

FIG. 7C is a graphical illustration of a number of cells detected on either side of a second microphysiological device after infection of the second microphysiological device and migration of the cells from a first microphysiological device, according to an exemplary embodiment of the present disclosure.

### DETAILED DESCRIPTION

The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language).

Reference throughout this document to “one embodiment”, “certain embodiments”, “an embodiment”, “an implementation”, “an example” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

In one aspect, the present disclosure describes a bottom-up approach towards in vitro emulation of human bone marrow. Methods described herein harness the regenerative capacity of adult stem cells to self-assemble a complex, specialized microenvironment of human hematopoietic stem cells in a vascularized three-dimensional microphysiological system. This in vitro bone marrow may be referred to herein as, among other terms, a micro-engineered bone marrow (MEBM) model or a micro-engineered niche.

The present disclosure includes demonstration through flow cytometry and single-cell transcriptomic interrogation that the micro-engineered niche, according to systems and methods described herein, can reconstitute hematopoietic stem cell self-renewal, multilineage differentiation, multilineage hematopoiesis, and/or complex ligand receptor signaling pathways of the native human marrow.

The abilities of the micro-engineered niche to generate functionally mature myeloid cells also makes it possible to mimic the key physiological processes of innate immunity including neutrophil chemotaxis and intravascular mobilization. To this end, the MEBM model of the present disclosure is evaluated via a model of bone marrow ablation by proton beam radiotherapy. Further, and to demonstrate the advanced application of “bone marrow-on-a-chip”, the microphysiological device of the present disclosure is evaluated within a multiorgan model of innate immune response against bacterial lung infection.

Altogether, the present disclosure advances the ability to reconstruct, probe, and deconvolve the complexity of the bone marrow niche, thereby enabling new capabilities to model human hematopoiesis and immunity for biomedical and pharmaceutical applications.

As background to the MEBM model introduced above, it can be appreciated that the present disclosure generally discloses techniques for producing a tissue, body organ or system, or an organ-on-chip using microfluidic devices. When a plurality of microfluidic devices is used together, the disclosed subject matter can perform a fully or partially automated organ culture using the organ-on-chip without the need for specialized personnel by modeling feed-forward and feedback effects from interfacing one functional unit to another in the organ-on-chip.

In certain embodiments, the MEBM model can include engineered vessel networks. For example, vascular endothelial cells, fibroblasts, pericytes, mesenchymal stem cells, and/or smooth muscle cells can be seeded together into a three-dimensional (3D) scaffold, such as an extracellular matrix scaffold or hydrogel, and supplied with culture medium. The culture medium may be endothelial cell media containing vasculogenic factors such as, among others, vascular endothelial growth factor (VEGF), fibroblast growth factor (FGF), and endothelial growth hormones. 3D fibrin hydrogel, collagen hydrogel, or other biocompatible hydrogel, and the like, or a combination thereof, can be used as the 3D scaffold. Often in the presence of the above-defined growth factors, the cells can form patterned vessel structures with hollow, perfusable lumens through the process of vasculogenesis, angiogenesis, or a combination of vasculogenesis and angiogenesis. The perfusable vasculature may comprise vessels having a hollow, endothelial cell-lined lumen and surrounded by pericytes or fibroblasts or a combination thereof in the surrounding stroma.

In certain embodiments, the MEBM model of the present disclosure can provide perfusion for the development, survival, regulation, and homeostasis of tissues by promoting blood vessels in the tissues. Blood can carry nutrients, oxygen, signaling hormones, various cell types including erythrocytes, platelets, leukocytes, and stem cells, as well as metabolic waste products and carbon dioxide. To permit perfusion as in human tissue, the MEBM model of the microphysiological device can have a similar vasculature as in the human body. For instance, the MEBM model may include vessels of a variety of sizes that model those in the human body, which can be branched from the large aorta leaving the heart (e.g., ˜20-30 mm diameter) through arteries (e.g., ˜0.1-10 mm diameter), arterioles (e.g., ˜0.01-0.1 mm diameter), and capillaries (e.g., ˜0.005-0.01 mm diameter), at which point diffusion and transport of bloodborne elements into and out of the surrounding tissue can occur. From the capillary networks, blood can circulate back through venules (˜0.008-0.1 mm diameter) and veins (˜0.1-15 mm diameter). Blood vessels can include an inner endothelium formed from endothelial cells, which are surrounded by innervated smooth muscle cells, vascular pericytes, and fibroblasts in connective tissue. In certain embodiments, the MEBM can provide reciprocal signaling that can occur between biological tissues and the vessels that perfuse it. Localized hypoxia or nutrient deprivation in human tissue can cause the secretion of signaling molecules including hypoxia induced factors (HIFs), FGFs, and VEGF. The secretion of signaling molecules can promote vasculogenesis, the formation of blood vessels from precursor cells, and angiogenesis, the sprouting of vessels from existing endothelial tissue.

Altogether, the MEBM model of the microphysiological device of the present disclosure seeks to mimic certain developmental processes of the human body. For instance, it can be appreciated that hematopoietic stem cell precursors arise from hemogenic sites during development and move through various embryonic niches in distinct anatomical locations that provide signals necessary for hematopoietic stem cell expansion and maturation. The original pool of hematopoietic stem cells generated by this complex, sequential process migrates to the bone marrow and seeds its nascent microenvironment towards the end of gestation. Importantly, the subsequent colonization of the bone marrow with hematopoietic stem cells occurs concurrently with the formation and maturation of sinusoidal blood vessels in the medullary cavity. Research has shown that this simultaneous process leads to the development of a specialized bone marrow niche required for the emergence of functional hematopoietic stem cells and the onset of their hematopoietic activity.

In the present disclosure, human hematopoiesis is emulated by reproducing this developmental process within a microphysiological microfluidic system. The microphysiological microfluidic system of the present disclosure, therefore, enables the engineering of vascularized human tissue constructs reminiscent of the hematopoietic vascular niche in vivo.

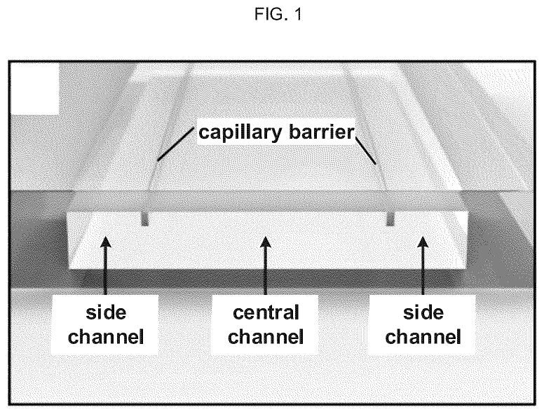

According to an embodiment, the MEBM model of the microphysiological device of the present disclosure may form a biomimetic analog of the human marrow. The microphysiological device may include one or more compartments, which may be referred to herein as channels. In an embodiment, the microphysiological device includes at least three parallel channels, as represented in FIG. 1. A capillary barrier may be formed between each of the side channels and the central channel of the microphysiological device or at an interface between each of the side channels and the central channel of the microphysiological device. The capillary barrier may allow for movement of fluids and the like between the channels of the microphysiological device.

As will be demonstrated, capillary barriers can be used for containment and/or control of liquids and liquid-based structures. In the present disclosure, capillary barriers limit the ability of a meniscus of a body of liquid to advance or recede within and between channels of the microphysiological device, thereby defining, in an instance, an interface between the channels.

In an embodiment, each capillary barrier is a structure within a volume of the microphysiological device and along a length of the microphysiological device. The structure may be formed on and/or within a surface of the volume of the microphysiological device. In embodiments, the structure may extend at least partially along the entire length of the microphysiological device. For instance, the length of the structure may be less than or equal to the length of the microphysiological device. It should be appreciated that reference to the length of the structure refers to a total length thereof, though it may be that the structure is discontinuous along that length. In other words, the structure may be a segmented series of structures. In embodiments, the structure may be proud or inferior to the surface of the volume of the microphysiological device. For instance, the structure may be a protrusion on the surface of the volume of the microphysiological device, may be a groove or depression within the surface of the volume of the microphysiological device, or a combination thereof. In an example, the structure is a protrusion on the surface of the volume of the microphysiological device. Pinning of the meniscus on the resulting structure requires such additional energy for the liquid meniscus to cross it that the liquid is confined unless additional energy is applied to the body of liquid.

In an embodiment, the capillary barriers separating the channels of the microphysiological device can be designed to permit controlled mixing, diffusion or perfusion of liquids, substances, and the like between the channels. This allows realistic scenarios in which, as in the MEBM model of the present disclosure, chemical signals, medium-derived nutrients, and the like can be transported between channels of the microphysiological device. This also means that cellular activities and responses within a first channel may be reactive to conditions within a second channel, as may be the case when a chemotactic agent or pathogenic material is present within the second channel and exposed to cellular matter within the first channel.

Further to the above, by providing the side channels adjacent the central channel of the microphysiological device, viability of cells in the central channel, as well as in the side channels, can be maintained. The side channels of the microphysiological device provide for, as an example, the transport of nutrients, oxygen, carbon dioxide, growth factors, other proteins, signaling molecules, compounds, further cells and the like into the central channel of the microphysiological device while allowing transport of waste products, metabolites, and the like away from the central channel.

In an embodiment, and as will be described later, the side channels of the microphysiological device may be connected in a fluid circuit to fluidly connect the microphysiological device with a supply/sink, a diagnostic module, a continuous flow module including a pump, or in a multi-organ circuit, wherein the multi-organ circuit comprises the microphysiological device, as a first microphysiological device, connected with a second microphysiological device modeling a different tissue, organ, and/or organ system.

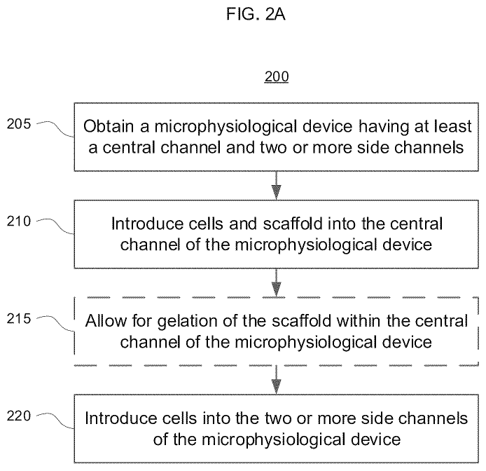

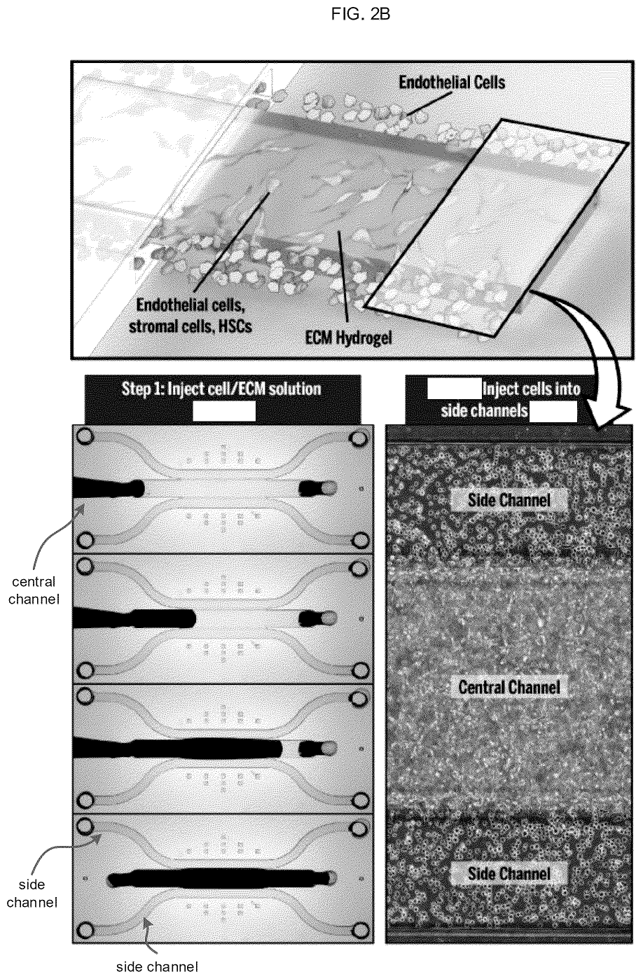

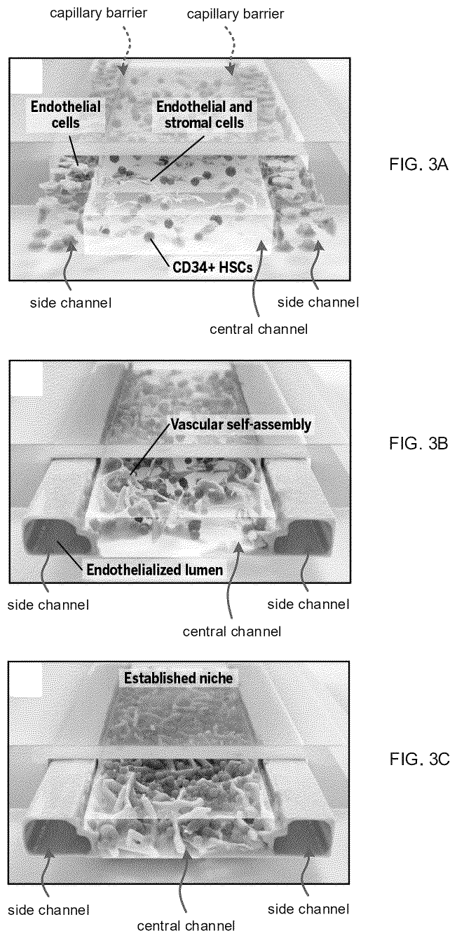

Returning now to the Figures, FIG. 2A through FIG. 3C describe development of the MEBM model of the present disclosure. In FIG. 2A, method 200 describes the initialization of the MEBM model within a microphysiological device.

At step 205 of method 200, a microphysiological device as described above and in FIG. 1 can be obtained.

In an embodiment, the obtained microphysiological device can be fabricated by, first, casting poly(dimethylsiloxane) (PDMS) onto micropatterned silicon-wafer molds manufactured in a cleanroom by typical photolithographic workflows in SU-8 negative photoresist. The cast PDMS can then be degassed in a desiccator vacuum chamber using house vacuum to remove trapped air. The cast wafer can then be placed in a convection oven overnight for curing. Following overnight curing, the cast PDMS can be cut from the wafer, fluidic access ports may be punched using a biopsy punch, and the cast PDMS may be trimmed to a rectangular form factor using a scalpel and bonded to the tissue culture plastic by contact electrostatic interaction to create a sealed microfluidic enclosure. A subsequent casting of PDMS can then be created with the same casting process as described above, but instead of casting the PDMS onto a micropatterned silicon-wafer mold, the PDMS can be cast onto an empty, un-patterned silicon wafer. The second cast PDMS can then be cut to the same rectangular shape as the first cast PDMS and punched with a biopsy to create reservoir holes at the same spacing as the fluidic access ports of the first cast PDMS. The second cast PDMS can then be mated with the first cast PDMS such that holes on opposing surfaces are aligned.

In an example, to create the micropatterned silicon-wafer molds, quartz photomasks coated with AZ1500 positive photoresist were patterned on a Heidelberg DWL66 Plus laser mask writer and developed with MF319 immersion for 80 seconds, followed by rinsing with water. The exposed nichrome film was etched in Chrome Etchant Type 1020 (Transene, Inc) for 120 seconds, rinsed with water, and stripped of photoresist by a 60 second immersion in Microposit Remover 1165 in a sonicated bath, after which they were rinsed sequentially with acetone, methanol, isopropanol, and blown dry with filtered air. This was repeated to create two photomasks. SU-8 2100 was spin coated to 200 μm thickness at 1500 RPM on a 6″ silicon wafer (prime grade), baked according to manufacturer datasheets (“SU8 2000 Processing Guidelines”, MicroChem, Inc.), exposed through the first photomask on a SUSS MA-6 mask aligner, and allowed to post-bake according to the manufacturer datasheets. Afterward, a second layer of SU8 2100 was spin coated to 200 μm at 1,500 RPM above the first exposed layer, the bake steps were repeated, and the wafer was exposed on the SUSS MA-6 mask aligner following optical alignment of the first exposed photoresist layer to the second photoresist mask. Following the second exposure, the wafer was post-baked a second time, was ramped down to room temperature (21° C.) over a 1-hour period, and then developed in SU-8 Developer by overnight immersion. After developing, the wafer was sequentially rinsed with acetone, methanol, and isopropanol, was blown dry with filtered compressed air, and was the coated overnight in a vacuum chamber with Trichloro-(1H, 1H, 2H, 2H-perfluorooctyl)-silane as a permanent release agent.

In an example and following fabrication of the micropatterned silicon-wafer molds, PDMS mixed at 10:1 ratio of monomer to curing agent by weight was cast over the micropatterned silicon-wafer molds. The cast PDMS was subsequently degassed in a desiccator vacuum chamber using house vacuum for 30 minutes to remove all bubbles. The cast PDMS was then placed in a 65° C. convection oven overnight to cure. Following overnight curing the mold was cut from the wafer, fluidic access ports were punched using a 1 mm biopsy punch, trimmed to a rectangular form factor using a scalpel, and bonded to the tissue culture polystyrene plastic of a round or rectangular petri dish by contact electrostatic interaction to create a sealed microfluidic enclosure. A second casting of PDMS was made with the same casting process, except poured over an empty, un-patterned silicon wafer to a height of 3 mm. The second PDMS cast was cut to the same rectangular shape as the first PDMS cast, using a scalpel, and punched with a 3 mm biopsy punch to create reservoir holes at the same spacing as the fluidic access ports of the first PDMS cast. This second cast of PDMS was then placed on top of the first, such that the larger holes on the second PDMS cast aligned with the inlet ports punched into the first PDMS cast to create reservoirs, and sealed by non-permanent PDMS-PDMS contact bonding in order to complete fabrication of the microphysiological device. The completed microphysiological device was then sterilized by placement into a cell culture hood and exposure of the microphysiological device to UV light for 30 minutes, thereby allowing UV light transmission through the PDMS.

At step 210 of method 200, cells and a scaffold can be introduced into the central channel of the microphysiological device. In an embodiment, the cells and the scaffold can be introduced separately. In an embodiment, the cells and the scaffold can be introduced concurrently. In some embodiments, the scaffold is introduced to the central channel of the microphysiological device as a precursor of a 3D scaffold. In this way, the precursor can be fluidly introduced into the central channel as a “pre-gel”. Moreover, this allows the cells to be mixed with the scaffold prior to being introduced into the central channel of the microphysiological device. In some embodiments, the scaffold is introduced to the central channel of the microphysiological device as a 3D structure, or a “gel”. In this way, cells can be subsequently introduced to the central channel of the microphysiological device and seeded into the 3D structure of the scaffold.

In an embodiment, the cells may include, among others, embryonic stem cells, induced pluripotent stem cells, hematopoietic stem cells, progenitor cells, mesenchymal stromal cells, fibroblasts, and endothelial cells. Additionally, the cells may include other cell types of diagnostic value or therapeutic value, such as cancer cells associated with bone marrow-related cancers, osteoblasts and related bone cells associated with bone maintenance, myeloid progenitor cells and/or lymphoid progenitor cells associated with the immune response and differentiation of hematopoietic stem cells, combinations of cells excreting hormones or other signaling factors, and other cell types and combinations thereof that are of interest. In embodiments, the cells may be myeloid cells and include myeloblasts, immature basophils, basophils, immature eosinophils, eosinophils, N. promyelocytes, N. myelocytes, N. metamyelocytes, N. bands, neutrophils, immature monocytes, monocytes, megakaryocytes, platelets, pronormoblasts, basophilic normoblasts, polychromatic normoblasts, orthochromatic normoblasts, polychromatic erythrocytes, and/or erythrocytes. In embodiments, the cells may be lymphoid cells such as lymphocytes.

In an embodiment, the cells may be derived from a variety of sources depending on the application of the microphysiological device. For instance, the cells may be derived from human, porcine, aquiline, giraffine, ursine, anserine, asinine, vulpine, feline, canine, murine, bovine, cameline, caprine, hircine, cervine, corvine, elephantine, formicine, hippotigrine, hyenine, leporine, lupine, macropine, octopine, ovine, piscine, ranine, taurine, tigrine, vespine, and vulturine, among others. In an example, the cells are derived from a human cell source.

In an embodiment, the scaffold may be a naturally-derived scaffold, a synthetic scaffold, or a combination thereof. In an embodiment, the scaffold may be degradable (biologically or otherwise) or non-degradable. Scaffolds can be selected based on the demands of the specific cellular tissue being investigated, since a variety of materials and techniques can be used to alter the scaffold characteristics.

In an embodiment, the scaffold may be a hanging drop scaffold, a hydrogel scaffold, a paper-based scaffold, a fiber-based scaffold, an additive manufacturing derived scaffold, and an electrospun scaffold, among others. In an embodiment, the scaffold may comprise components of extracellular matrix. In an embodiment, the scaffold may comprise collagen (e.g., type I collagen), Matrigel™ (or similar basement-membrane matrix), polylactic acid, polyglycolic acid, poly(lactide-co-glycolide), poly-(&-caprolactone), chitin, fibrinogen, alginate, agarose, cellulose, gelatin, PDMS, polyethylene glycol, and polyurethane, among others.

In an embodiment, the cells may include hematopoietic stem cells, mesenchymal stromal cells, and endothelial cells and the scaffold may include extracellular matrix components. The cells may be obtained from a human source. The scaffold may be obtained from a human source or from another source but stripped of its immunogenic features.

Returning to method 200, in an exemplary embodiment of step 210, an extracellular matrix “pre-gel” combined with cells, including human hematopoietic stem cells, endothelial cells, and mesenchymal stromal cells, can be introduced into the central channel of the microphysiological device to create an extracellular matrix (ECM) hydrogel construct containing the cell mixture.

In an example, the cells are combined with the ECM hydrogel are selected to recapitulate human bone marrow (i.e., the MEBM model of the microphysiological device). To this end, the cells include human endothelial cells (HUVECs), mesenchymal stromal cells (MSCs), and hematopoietic stem cells (HSCs). In certain embodiments, the cells include fibroblasts. To obtain adequate cell numbers, the HUVECs, MSCs, and fibroblasts were seeded into adherent tissue culture plastic in Corning TC-treated T-75 tissue culture flasks and cultured in endothelial growth media (HUVECs: Lonza EGM-2) or fibroblasts media (MSCs, fibroblasts; Lonza FGM-2), respectively, and used after one passage for formulating the MEBM model. CD34+ HSCs were obtained from de-identified human donors following whole marrow extraction from the iliac crest and subsequent purification of HSCs by CD34-based positive selection with immunomagnetic microspheres. CD34+ HSCs were placed into suspension culture in serum-free expansion media (SFEM II, StemCell Tech. Cat. 09605) supplemented with CC100 (StemCell Tech. Cat. 02690).

In an example, the procedure for introducing the cells and the scaffold into the microphysiological device included preparation of the cell-based ECM hydrogel mixture. An ECM precursor solution, or “pre-gel”, may be made by suspending each cell type (endothelial cells, fibroblasts, MSCs, and CD34+ HSCs cells) in a solution including fibrinogen in saline and Matrigel™. In particular, the ECM precursor solution may be made by suspending each cell type at 2.5×10⁶ cells/ml in a solution made from mixing, at 1:1 by volume, (1) 11.11 mg/ml fibrinogen in phosphate buffered saline (PBS) (e.g., Dulbecco's PBS) and (2) growth factor reduced Matrigel™. The solution was maintained on ice in 180 μl aliquots. Immediately prior to injection and while still cold, the solution was mixed with 20 μl of 10 U/ml thrombin (final concentration 1 U/ml) and then injected directly into the central channel of the microphyisological device being seeded, as shown in FIG. 2B. The resulting microphysiological devices, having been seeded with liquid precursor solution, were placed in an incubator at 37° C. for 20 minutes for gelation (at step 215 of method 200) of the composite hydrogel to occur.

Step 215 of method 200 is enclosed by a dashed rectangle to indicate that gelation is required only in accordance with a type of scaffold used in the central channel of the microphysiological device.

In an embodiment, at step 220 of method 200 and after gelation of the scaffold within the central channel of the microphysiological device, the side channels flanking each side of the central channel can be filled with establishment media (see Table 1) and cells, as shown in FIG. 2B.

In an embodiment, the cells may include, among others, embryonic stem cells, induced pluripotent stem cells, hematopoietic stem cells, progenitor cells, mesenchymal stromal cells, fibroblasts, and endothelial cells. Additionally, the cells may include other cell types of diagnostic value or therapeutic value, such as cancer cells associated with bone marrow-related cancers, osteoblasts and related bone cells associated with bone maintenance, myeloid progenitor cells and/or lymphoid progenitor cells associated with the immune response and differentiation of hematopoietic stem cells, combinations of cells excreting hormones or other signaling factors, and other cell types and combinations thereof that are of interest. In embodiments, the cells may be myeloid cells and include myeloblasts, immature basophils, basophils, immature eosinophils, eosinophils, N. promyelocytes, N. myelocytes, N. metamyelocytes, N. bands, neutrophils, immature monocytes, monocytes, megakaryocytes, platelets, pronormoblasts, basophilic normoblasts, polychromatic normoblasts, orthochromatic normoblasts, polychromatic erythrocytes, and/or erythrocytes. In embodiments, the cells may be lymphoid cells such as lymphocytes.

In an embodiment, the cells may be derived from a variety of sources depending on the application of the microphysiological device. For instance, the cells may be derived from human, porcine, aquiline, giraffine, ursine, anserine, asinine, vulpine, feline, canine, murine, bovine, cameline, caprine, hircine, cervine, corvine, elephantine, formicine, hippotigrine, hyenine, leporine, lupine, macropine, octopine, ovine, piscine, ranine, taurine, tigrine, vespine, and vulturine, among others. In an example, the cells are derived from a human cell source.

In an embodiment, the cells within the side channels of the microphysiological device are endothelial cells. In an example, the cells within the side channels of the microphysiological device may include HUVECs. In embodiments, the endothelial cells may be vascular endothelial cells, lymphatic endothelial cells, or a combination thereof. An exemplary seeded microphysiological device is shown in FIG. 2B.

TABLE 1
Media Formulations

| Media | | |
|---|---|---|
| Formulation | | Experimental |
| Name | Media Formulation Contents | Usage |
| Establishment | StemCell SFEM II Base | Days 0-6 in |
| Medium | supplemented with 1x StemCell | device culture, |
| | CC100, rhVEGF (0.5 ng/ml), | where Day 0 is |
| | bFGF (5 ng/ml), IGF-1 R3 | the day on |
| | (10 ng/ml), Ascorbic Acid | which the |
| | (1 μg/ml), EGF (1 ng/ml) | devices are |
| | | seeded. |
| Erythropoiesis- | StemCell SFEM II supplemented | Days 7-21 |
| Specific | with rh VEGF (0.1 ng/ml), | |
| Medium | bFGF (1 ng/ml), IGF- | |
| | 1 R3 (2 ng/ml), Ascorbic | |
| | Acid (0.2 μg/ml), EGF (1 ng/ml), | |
| | EPO (1 ng/ml), Flt3 (10 | |
| | ng/ml), and IL3 (5 ng/ml) | |
| Myelopoietic | StemCell SFEM II supplemented | Days 7-21 |
| Medium | with rhVEGF (01. Ng/ml), | (alternative to |
| | bFGF (1 ng/ml), IGF- | erythropoiesis- |
| | 1 R3 (2 ng/ml), Ascorbic | specific |
| | Acid (0.2 μg/ml), EGF (1 ng/ml), | medium) |
| | EPO (1 ng/ml), Flt3 (10 | |
| | ng/ml), IL3 (5 ng/ml), | |
| | G-CSF (0.1 ng/ml) | |
| Circulation | StemCell SFEM II supplemented | During |
| Medium | with rhVEGF (01 ng/ml), | multi-organ |
| | bFGF (1 ng/ml), IGF-1 | perfusion. |
| | R3 (2 ng/ml), Ascorbic | |
| | Acid (0.2 μg/ml), | |
| | EGF (1 ng/ml) | |

An illustration of method 200 of FIG. 2A is shown in FIG. 2B. The lower panel images provide aerial views of the isometric perspective of the microphysiological device of the upper panel image. The lower left panel images illustrate the introduction of the cells and the scaffold at step 210 of method 200. The lower right panel images illustrate the microphysiological device after having introduced cells into the side channels at step 220 of method 200.

After performing method 200 of FIG. 2A, in vitro development of the MEBM model of the microphysiological device continues in the presence of the above-described media formulations. This development includes, as shown in FIG. 3A through FIG. 3C, which reflect time-dependent development of the MEBM model of the microphysiological device, performance of method 200, vascular self-assembly, and endothelialization of the side channels of the microphysiological device, resulting in an established bone marrow niche in FIG. 3C. In particular, FIG. 3A depicts initial cell seeding (i.e., Day 1), FIG. 3B depicts neovascularization of the scaffold (i.e., Days 2-7), and FIG. 3C depicts dense colonization of HSCs around a matured vascular network (i.e., Days 7-14).

In other words, over time, the 3D co-culture configuration in the MEBM model of the microphysiological device induces vasculogenic self-assembly of endothelial cells, as shown in FIG. 3B, leading to de novo formation of a network of blood vessels that approximate the sinusoidal vasculature of native bone marrow. Via the capillary barriers shown in FIG. 1 and indicated by the dashed arrows of FIG. 3A, these newly formed vessels may anastomose with the endothelial lining of the side channels, making them directly accessible and perfusable from the side channels. In this way, and in an embodiment, each of the side channels represent one of an arterial component and a venous component of the bone marrow. During this progressive process of vascular development, the hydrogel scaffold may also be supplied with soluble factors that support the maintenance of the embedded HSCs and allow them to populate the perivascular regions of the construct o recapitulate HSC colonization during the development of the hematopoietic vascular niche in vivo, as shown in FIG. 3C.

Evaluations of the microphysiological device of the above-described Figures, as well as perturbations and evaluations thereof, will be described in detail below.

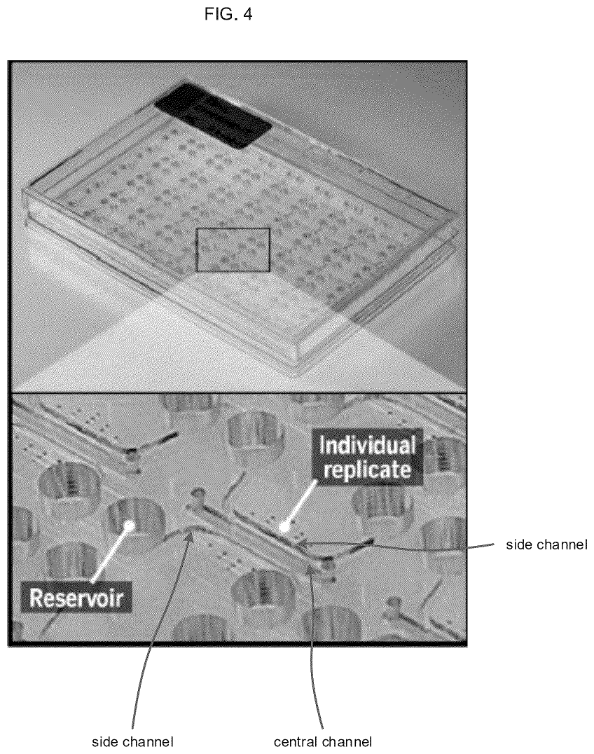

It can be appreciated that the above discussion of the microphysiological device has been limited to a single microphysiological device. It can also be appreciated that a plurality of microphysiological devices may be deployed within a μ-well plate-based form factor to increase the scale at which development, observations, and analysis of the MEBM model may occur. For instance, as in FIG. 4, micro-engineered models of the MEBM may be scaled in throughput with a plate-based form factor that contains, in an example, 40 independent replicates, which can be seeded, cultured, and otherwise maintained by a fluid handling robot. As in the lower image of FIG. 4, which is a magnified view of a single microphysiological device and portions of other, neighboring microphysiological devices, it can be appreciated that each microphysiological device includes reservoirs directly connected to side channels of the microphysiological device and a central channel therebetween.

In an embodiment, the microphysiological device can be cultured on a custom-built plate tilting robot. Plates can be left on the tilter for the duration of experimentation except for media replacements on every second day. In an embodiment, media replacements can be performed robotically by a fluid handling robot.

### Proof of Concept Demonstration

According to an embodiment, the MEBM model and microphysiological device described above was deployed for a proof-of-concept demonstration. To this end, the microphysiological device was used to co-culture CD34+ human HSCs with primary HUVECs, fibroblasts, and MSCs in an ECM hydrogel comprised of fibrin and Matrigel™. To increase experimental throughput, the above-introduced multi-well plate design was implemented to create a device containing an array of 40 individually addressable microfabricated cell culture units, the multi-well plate design being directly compatible with robotic fluid handling and automated imaging.

Within 2 days of seeding, the endothelial cells began to establish intercellular connections and subsequently formed a capillary network of interconnected vascular tubes over a period of 5 days. The self-assembled vessels became perfusable after 7 days of culture as a result o their anastomosis with the endothelium in the side channels. Interestingly, intravascular flow was found to stabilize vascular architecture and inhibit pruning of blood vessels, which supports the maintenance of a dense vascular network during prolonged culture.

Importantly, the vascularization of the MEBM model of the microphysiological device was accompanied by a substantial increase in the activity of HSCs. During the seven-day progression of vasculogenesis, CD34+ HSCs were fed with CC100 containing serum-free media to support stem cell proliferation and maintenance. Under this condition, the starting population of HSCs underwent rapid expansion and began to form densely populated colonies within 14 days. The multicellular clusters were visible throughout the scaffold and continued to grow over time, indicating successful colonization of the vascularized construct with HSCs.

Having confirmed the vascularization and HSC colonization of the micro-engineered niche, the potential to deploy the MEBM model to reconstitute multilineage hematopoiesis was investigated. Considering that hematopoiesis in vivo relies on extrinsic cues to control the fate of HSCs, the hematopoietic investigation was conducted by the addition of soluble factors known to drive lineage differentiation. First, the MEBM model was treated with erythropoietin (EPO) (see “Erythropoiesis-Specific Medium” of Table 1) to stimulate erythropoiesis. The addition of EPO led to the formation of multicellular complexes within the micro-engineered niche, each of which consisted of a central macrophage covered with erythroid cells that resembled erythroblastic islands in vivo. Following this phenotypic organization, the MEBM model produced a substantial number of round, erythroid cells that were distinguishable by their red color and expression of transferrin receptor (i.e., CD71) and glycophorin A. The micro-engineered niche also supported the differentiation of HSCs into myeloid cells. Stimulation of the MEBM model with myelopoietic media (see Table 1) for 21 days produced a marrow construct densely packed with cells of myeloid lineages, most of which were situated proximate the vasculature. A significant number of the cells were terminally differentiated mature neutrophils as identified by their multisegmented nuclei, intracellular secretory granules, and surface marker expression (CD15+/CD66b+). Immunofluorescent, flow cytometric, and transmission electron microscopy analysis revealed that the differentiated cell population also contained other types of granulocytes such as eosinophils, macrophages, and monocytes, thus approximating the repertoire of immune cell types generated by myelopoiesis in vivo.

Importantly, the MEBM model retained stem cell population even during extended periods of myelopoietic differentiation. This was evidenced by the presence of CD34+ cells throughout the scaffold after 28 days of culture, which is in contrast to rapid loss of self-renewing HSCs within approximately 5 days in traditional cell cultures. This data also showed multiple lineages of myeloid progenitor cells in the same scaffold. Taken together, these results demonstrate the feasibility of engineering a complex and biologically active hematopoietic milieu that can emulate key features of the bone marrow vascular niche.

### Airway-On-a-Chip

An airway-on-a-chip was fabricated similarly to that described in U.S. patent application Ser. No. 15/748,039, which is incorporated by reference herein in its entirety.

Briefly, master molds for a model of distal airways were fabricated according to the same photolithographic procedures used for the MEBM model molds, described above. The airway model was fabricated from three layers of PDMS, each separated by a permeable membrane disk (3.0 μm pores) punched from PET transwell filters with an 8 mm biopsy punch (Integra). The stack of PDMS and permeable membranes were bonded using uncured PDMS as a mortar.

Following fabrication, the device consisted of three chambers in sequential contact. First, an apical chamber that represented the human airway was seeded with human small airway epithelial cells at 2×10⁶ cells/ml in Lonza SAGM growth media. Next, a central channel that represented the interstitium was seeded with 1 mg/ml collagen type I hydrogel containing normal human lung fibroblasts at 1×10⁵ cell/ml. Lastly, a basal chamber was coated with fibronectin and collagen type I and subsequently seeded with pulmonary microvascular endothelial cells at a concentration of 2×10⁶ cells/ml.

After 1 day of submerged culture, the small airway cells were taken to air-liquid interface (ALI), and the media in the basal chamber was changed to contain, by volume, 80% StemCell Pneumacult ALI Medium formulated as specified by the manufacturer and 20% Lonza EGM-2 MV with 5× the recommended supplement concentration (to yield a 1× final supplement concentration). This media was used to nourish the cells for an additional 20 days (total 21 days post-seeding until deemed mature for functional experimentation), with media changes performed every second day.

ALI culture was accomplished by flowing a mixture of culture media through the basal chamber, or lower vascular chamber, and feeding the epithelial cells of the apical chamber from the basolateral side through the hydrogel and the intervening membranes. This conditioning permitted epithelial differentiation over a period of three weeks without loss of cell viability to produce an in vivo-like airway epithelium with beating cilia and a continuous network of intercellular tight junctions. The differentiated phenotype of the engineered tissue was further evidenced by the ultrastructure of the epithelial layer visualized by transmission electron microscopy (TEM). The underlying interstitium showed fibroblasts uniformly distributed throughout the hydrogel scaffold in close opposition to a confluent endothelial monolayer.

In one evaluation of the airway models, immunostaining or electron microscopy fixatives were carefully flowed into the microphysiological devices, gently displacing the contained culture media to avoid dislodging immune cells with excessive fluid shear on the airway model surfaces. For both immunofluorescent and electron microscopy, in their respective fixation buffers, fixation was performed overnight at 4° C. Following overnight fixation, the devices were rinsed repeatedly with PBS over a 4-hour period. Immunohistological staining was performed within the airway devices following the same protocols as the MEBM models. After staining, the chips were peeled apart, and the semipermeable membranes containing the epithelial and endothelial tissues were carefully removed with tweezers and mounted onto slides with curing mountant solution (ProLong Diamond, ThermoFisher P36965). For electron microscopy, the same protocol was followed except without staining; the membranes were removed, dehydrated in an ethanol series as previously described for the bone marrow chips, dried at the CO2 critical point (Tousimis Autosamdri-810), sputtered with 60/40 palladium-gold alloy, and then imaged on an FEI Quanta 250 FEG scanning electron microscope.

As described in subsequent sections, the airway-on-a-chip will be referred to as a second microphysiological device.

### Experimental Methods and Results

i. Interrogating the Engineered Hematopoietic Niche at Single Cell Resolution

A single-cell RNA sequencing (scRNAseq) was to identify different cell populations present in the MEBM model of the microphysiological device and to interrogate gene expression profiles. This allows for a deeper understanding into the complexity of the engineered vascular niche and its hematopoietic activity.

In the study, 20 replicates of the MEBM model of the microphysiological device were generated from human HSCs derived from the same donor. The replicates of the MEBM model were cultured for 3 weeks. Each of the MEMB models were then isolated and processed for mRNA library preparation and sequencing.

An overview of cellular composition generated by uniform manifold approximation and projection (UMAP) analysis showed that the micro-engineered niche of the MEBM model encompassed many cell types that play a critical role in regulating the hematopoietic microenvironment of HSCs. In addition to PECAM1+/CDH5+ endothelial cells comprising the blood vessels, a large cluster of cells expressing leptin receptor (LEPR) and collagen type I α1 (COL1A1) were observed, which have been suggested as specific markers of perivascular stromal cells in the native marrow. These cells overlapped substantially with those expressing CXC chemokine ligand 12 (CXCL12), a multifunctional niche factor essential for maintaining the hematopoietic niche. Within the stromal cell cluster, subpopulations of cells that exhibited transcriptomic signatures of perivascular mesenchymal stem cells, including pleiotrophin, CD44, CD73, and CD90, were also identified. Detection of the RUNX2 transcription factor in the same cluster indicated the presence of osteoblast lineage cells in the stromal cell population, which represent the key endosteal components of the HSC niche in vivo. The expression of marker genes that delineated a wide range of cell subpopulations was observed.

Myelopoiesis in the MEBM model was evident from the presence of S100A9+ cells that contained transcriptomically-distinct populations of myelopoietic progenitors and HSC-derived progeny, including neutrophils, eosinophils, monocytes, and macrophages. Consistent with immunofluorescence analysis, the sequencing data showed the existence of HSCs in the micro-engineered niche as represented by a small cluster of cells positive for KIT. Both the myeloid cell population and HSCs displayed robust expression of CXC-chemokine receptor 4 (CXCR4) that mediates CXCL12 signaling responsible for retention of HSCs and hematopoietic progenitor cells in the bone marrow niche. Similarly active were signaling factors that contribute to the development and stabilization of blood vessels required for vascularization of the hematopoietic niche. This was illustrated by reciprocal signaling of angiopoietins (ANGPT1, ANGPT2) and vascular endothelial growth factors (VEGFA, VEGFB) between the perivascular stromal cells and endothelial cells.

Further to the above, scRNA-seq analysis was used to examine donor-dependent variability in the transcriptomic profiles of the engineered vascular niche. To this end, another set of 20 MEBM model replicates were generated using CD34+ cells derived from a different donor. The MEBM model replicates were subjected to the same culture conditions that induced myelopoietic differentiation. Comparison of the two groups representing two different donors revealed a nearly identical distribution of key cell types in UMAP. A substantial degree of similarity was also noted in the expression of cell type-specific markers and their spatial clustering, demonstrating the capability of the MEBM model of the microphysiological device to produce a functional hematopoietic niche in a reproducible manner.

Finally, a single-cell trajectory analysis was performed using the Monocle package to profile the dynamic process of myelopoiesis in the MEBM model. In this study, differentially regulated genes correlated with cell-state transitions from HSCs to myeloid lineages were detected and top-ranked genes were plotted against pseudotime, representing the progression of HSC differentiation. The data identified azurocidin 1 (AZU1) as a transcriptional marker with the highest positive correlation with the development and maturation of neutrophils, which is consistent with its established role as a key marker of terminally differentiated neutrophils. At late stages of differentiation, marked upregulation of genes known to encode proteins in primary granules of mature neutrophils, including cathepsin G (CTSG), serine protease 57 (PRSS57), myeloperoxidase (MPO), and neutrophil elastase (ELANE), was also observed. Similarly, the analysis yielded transcripts positively correlated with the progression of HSC differentiation into monocytes and macrophages, such as MS4A4A, CD163, and macrophage receptor (MARCO). Compared to neutrophil markers, most of these genes were activated much earlier and tended to peak in the middle of differentiation, after which their expression levels remained elevated. Transcriptional dynamics of eosinophilic development was associated with upregulation of CD24, eosinophil peroxidase (EPX), CLC, and CCAAT enhancer-binding protein epsilon (CBEPE). Increasing expression of these genes required for terminal differentiation of eosinophils was also accompanied by early downregulation of stem and progenitor cell markers such as KIT.

To perform the single cell mRNA sequencing described above, 20 independent MEBM model tissues for each scRNA Seq sample, of the two used above, were pooled and dissolved whole in 0.25% trypsin solution. The cells were washed twice with DPBS, libraries were generated with a 10X Chromium hardware using Single Cell 3′ v3 chemistry, and sequencing was performed on an Illumina NovaSeq to a depth of 25,204 mean reads per cell following aggregation of the two samples using the 10× Genomics Cell Ranger pipeline. Following output of barcoded count matrices from the 10× Genomics Cell Ranger-aggr pipeline, the data was imported into Seurat72 in the R environment, processed for QC of low-quality cells and doublets using typical QC metrics (e.g., high relative mitochondrial mRNA expression and barcodes with exceedingly high total transcripts), and then imported into Monocle336 for UMAP projection, clustering, gene expression analysis, and pseudotime trajectory analysis. QC-failed cells were preserved in the dataset during UMAP projection in order to capture other QC-cells that were at the decision boundaries during manual QC windowing and, thus, prevent their inclusion in the subsequent analysis. The QC-cluster (which self-selected>98% QC-cells identified by manual gating) was removed from all analysis, along with all remaining QC-cells identified manually by histogram gating that were distributed among the other clusters (<2%), primarily doublets.

ii. Probing Ligand-Receptor Interactions in the Engineered Hematopoietic Niche

In vivo, the bone marrow relies on the cooperation of multiple cell types regulated by complex intercellular signaling pathways to perform its specialized function as the principal hematopoietic organ. In this study, the MEBM model was evaluated to determine its capacity to emulate the complexity of the coordinated, context-dependent crosstalk between the cellular constituents of the hematopoietic vascular niche. To address this question, a comprehensive, systematic analysis of intercellular communication networks using the single-cell transcriptomics data was conducted.

Specifically, a computational package and public repository of ligands, receptors, and their interactions, called CellPhoneDB40, was used to identify enriched ligand-receptor interactions from the scRNA-seq datasets that were involved in soluble factor-mediated signaling between the subpopulations of cells in the engineered niche. Through pairwise comparisons of receptor and ligand expression between all cell types, this analysis yielded over 800 ligand receptor interactions that exhibited statistically significant (p<0.05) cell-type specificity. Mapping of p-values indicated that the identified ligand-receptor pairs were not ubiquitously expressed in the MEBM model and that significant interactions occurred in a highly cell type dependent manner. For closer examination, a subset of these data with a high degree of statistical significance (p<0.001) were plotted as chord diagrams to visualize the cell type-specific ligand-receptor pairs and their directional relationships predicted by the analysis. These diagrams show a number of physiologically relevant molecular pathways known to mediate cell-cell communication in the native hematopoietic niche. For example, KIT ligand (KITLG; also known as stem cell factor, SCF) secreted by endothelial and perivascular stromal cells interacted with KIT expressed by HSCs. Both paired KIT-KITLG interactions (endothelial-HSC, perivascular stromal-HSC) have been shown as critical requirements for HSC maintenance in vivo. The results also revealed endothelial production of Notch ligands (DLL1, JAG1) and their binding to Notch1 on HSCs, which plays an important role in the regulation of HSCs and hematopoiesis in vivo. Similar directional signaling between niche cells and HSCs/progenitors was observed for other well-characterized ligand receptor pairs, including CXCL12-CXCR4, growth and differentiation factor 11 (GDF11)-transforming growth factor receptor (TGFR), and bone morphogenetic proteins (BMPs)-BMP receptors (BMPRs). Also clearly represented was the crosstalk between the differentiated myeloid and endothelial/stromal cell populations, as exemplified by the interaction of colony stimulating factor 1 (CSF1) produced by perivascular stromal cells with CSFIR on macrophages. The proangiogenic endothelial-stromal crosstalk implicated in bone marrow development, such as FGF2-FGFR1 interactions, was also observed. These results demonstrate the capabilities of the engineered niche to recapitulate some of the key multicellular interactions present in the specialized microenvironment of the native marrow, supporting the physiological relevance of the MEBM model.

To perform the above-described receptor-ligand interaction analysis, CellPhoneDB package's Docker image (obtained from https://hub.docker.com/r/ydevs/cellphonedb) was obtained and a count matrix and cell-identity matrix consisting of the subpopulations annotated by canonical markers of cell identity and maturity was provided as an input. To narrow the scope of the analysis to identify key, bone marrow-specific interactions between stromal and hematopoietic/myeloid populations in the MEBM model of the hematopoietic niche, the CellPhoneDB output matrix of significantly interacting ligand-receptor pairs was filtered to remove all direct integrin-ECM interactions. Only the remaining interactions for which p<0.001 were preserved. For remaining interactions, chord plots of directional interactions between ligand →receptor were prepared using the Circlize package in R. This was enabled by the high spatial resolution to which the plethora of individual cell populations within the scRNA Seq dataset were able to be distinguished.

iii. Mobilization of Blood Cells in the Engineered Hematopoietic Niche

When hormones, cytokines, and other soluble signals produced by peripheral tissues reach the bone marrow, they can induce mature blood cells in the hematopoietic niche to transmigrate across the sinusoidal endothelium and enter the bloodstream. This regulated release of blood cells into circulation is termed mobilization, which represents a critical step in immunity and continuous cellular replenishment required for homeostasis of the hematopoietic system. Having validated the transcriptomic signatures of mature hematopoietic cells in the MEBM model and their capacity for physiologically relevant cell-cell signaling, the MEBM model was evaluated to determine whether it could reconstitute the complex process of mobilization in the integrated context of the vascularized hematopoietic niche. The study was specifically focused on investigating the ability of mature neutrophils differentiated from HSCs in the MEBM model to replicate rapid release of their in vivo counterparts from the bone marrow reserve during immune responses to inflammatory insults. To simulate this situation, the vasculature of the micro-engineered niche was perfused with culture media containing physiologically relevant concentrations of interleukin-8 (IL-8) during infection (5,000 μg/ml), a systemic inflammatory cytokine known to stimulate neutrophil release.

In control experiments without IL-8, the vast majority of mature neutrophils identified by their expression of CD66b were retained in the perivascular space. When IL-8 was infused, however, substantial egress of CD66b+ cells from the stromal compartment into the vascular lumen was observed. Intravascular translocation of these cells occurred rapidly, within one hour of treatment, consistent with the time scale over which neutrophils are mobilized from the native marrow in an inflammatory setting. These observations were further supported by scanning electron micrographs showing intravascular migration and intravasated myeloid cells found inside of the blood vessels that exhibited the typical morphology of granulocytes circulating in the blood.

To capture the process of mobilization under the dynamic conditions of continuous vascular perfusion, a time-lapse imaging of neutrophils in the micro-engineered niche was performed at physiological flow rates (10 μl/min). In the IL-8-treated replicates, neutrophils were distributed in the perivascular region initially, but upon cytokine stimulation, a large fraction of these cells entered the neighboring blood vessels in less than 20 minutes. The intravasated cells were then quickly cleared from the observation area in the direction of flow. The cellular content of effluent collected from these devices was composed primarily of non-adherent cells characterized by multilobular nuclei and robust expression of CD66b, confirming the release of terminally differentiated mature neutrophils. During the same time period only a few neutrophils were mobilized in the control group. A majority of the cells in this case remained in the stroma and showed undirected, random motility in their vicinity without migrating across the endothelial barrier.

The MEBM model was also responsive to AMD3100 (Plerixafor), a CXCR4 antagonist clinically used for mobilizing HSCs and other hematopoietic cells from the bone marrow for autologous transplantation. In comparison to untreated controls, the constructs perfused with a clinical dose of AMD3100 (25 μg/ml) showed a more than fivefold increase in the rate at which cells were released from the device. However, the rate of mobilization in this group was still lower than that in the IL-8-treated model. Image cytometry analysis of CD15 and CD66b staining revealed that approximately 65% of the cells released by AMD3100 were of myeloid lineage, 56% of which were neutrophils. This result was substantially different from that of IL-8-induced mobilization in which mature neutrophils represented the predominant (76%) population of released cells. These data illustrate the capacity of IL-8 to induce more rapid and selective mobilization of neutrophils from the bone marrow, which plays an essential role in innate immunity that serves as the first line of host defense. Given that neutrophil egress in the marrow entails complex, coordinated behavior of terminally differentiated cells, this demonstration of stimulated cell trafficking suggests functional maturity of HSC-derived neutrophils produced by the engineered niche.

For further investigation, a Boyden chamber migration assay was conducted to measure chemotaxis of mobilized neutrophils in a gradient of IL-8 as a representative endpoint of their functional capacity. The results showed that almost 80% of the cells moved across the membrane over a period of an hour, verifying the chemotactic function of the neutrophils. Migratory cells were still observed in the absence of the chemoattractant, but they accounted for a significantly (p<0.0001) smaller fraction of the population. It was noted that the transmigrated cells in the lower chamber clumped together to form multicellular aggregates, reminiscent of neutrophil swarming in vivo that plays an important functional role in antimicrobial activities of neutrophils.

iv. Modeling Bone Marrow Ablation by Proton Therapy

High sensitivity to ionizing radiation is a well-documented property of the bone marrow. A large body of research has demonstrated that exposure to high-energy electromagnetic or particle radiation can exert deleterious effects on the hematopoietic microenvironment of the human marrow, causing both hematopoietic and vascular pathology or a complete collapse of the niche. During cancer radiotherapy, for example, off-target exposure to ionizing radiation has been shown capable of damaging mature blood cells as well as hematopoietic stem and progenitor cells, which can lead to bone marrow injury and suppressed hematopoiesis. Chronic exposure to cosmic radiation is another instance that has been associated with increased risk of bone marrow dysfunction and malignancies in astronauts on prolonged missions.

Currently, investigating responses of the human marrow to these types of radiation exposures relies predominantly on the use of animal surrogates. Therefore, a preliminary study was conducted to explore whether the MEBM model of the microphysiological device could be used for modeling the effect of physiologically relevant radiation exposure on the hematopoietic vascular niche of the human marrow. The specific focus of this study was on creating an in vitro model of radiation induced bone marrow ablation, a common clinical procedure that utilizes high-dose ionizing radiation to eliminate hematopoietic cells prior to allogenic bone marrow transplantation. To establish this specialized model, a pencil beam scanning proton therapy system for controlled irradiation of the bone marrow-on-a-chip was employed. Importantly, the high spatial precision of pencil beam scanning was exploited in conjunction with computed tomography (CT) to create a 3D dose plan that permitted differential exposure of an array of engineered marrow tissues to a range of radiation doses spanning two orders of magnitude. Accurate delivery of proton radiation to the living constructs in the microphysiological device was validated by radiochromic film dosimetry and an array ionization chamber device.

Using this setup, radiation effects on the micro-engineered niche maintained in myelopoietic conditions were examined. For quantitative analysis, the number of CD15+ myeloid cells were measured over a period of two weeks after irradiation to assess the progression of bone marrow failure. At 0.2 Gray (Gy), the cellularity of the exposed constructs remained largely unchanged over the course of measurement, in contrast to the gradually increasing cell number in non-irradiated controls. However, exposure to 2 Gy resulted in a rapid, significant decrease in the number of CD15+ cells within 6 days of irradiation. This initial response was followed by a further decline in the cell density, yielding more than 65% reduction by the end of the 14-day period. At this point, the number of remaining myeloid cells was comparable to that measured in the positive control group irradiated with 20 Gy, which was confirmed by immunofluorescence imaging of the exposed tissues and scanning electron micrographs of injured myeloid cells in the perivascular space. The significant hematoablative effects following 2 Gy radiation exposure demonstrated by these results are in good agreement with the typical therapeutic window (2-3 Gy) of radiation used for bone marrow ablation in humans.

Studies have shown that irradiation causes collateral damage to the bone marrow microenvironment by disrupting the sinusoidal vasculature and generating free radicals and danger signals that have adverse effects on the stromal components of the hematopoietic vascular niche. Indeed, irradiation of the MEBM model of the microphysiological device at a therapeutic dose of 2 Gy compromised endothelial barrier function of the blood vessels, as illustrated by rapid leakage of intravascular dye into the perivascular space.

Based on these observations, bulk RNA sequencing (RNA-seq) of the irradiated marrow constructs was performed to examine the transcriptomic signatures of acute radiotoxicity in the engineered hematopoietic niche. To assess the variability of the data, principal component analysis of the RNA-seq data was performed, which yielded tight, dose dependent clustering of the differentially irradiated groups. Differential expression analysis showed substantial downregulation of differentiated cell markers across several lineages obtained in the MEBM model. Importantly, a sharp increase in the expression of genes implicated in stress and inflammatory responses was also observed.

A number of recent studies have suggested that ionizing radiation used in cancer therapies can induce immune dysregulation characterized by T helper type 2 (Th2)-skewed immune responses. These data showed that Th2 responses may limit the tumoricidal effects of radiation, but they may improve bone marrow recovery and function. While most of these studies have been conducted in murine models, evidence has demonstrated the occurrence of Th2 responses in human patients treated with ionizing radiation. The mechanisms by which this occurs, particularly in humans, remain obscure. Therefore, radiation of the MEBM model of the microphysiological device was used as a test case to discern whether mechanisms of Th2 immune skewing could be determined. Expression of several genes involved in immune regulation were elevated by irradiation of the MEBM model. The largest fold-change occurred in TNFSF15 (TIA, >32-fold increase, p<0.00001), which acts through its receptor DR3 on type 2 innate lymphoid cells (ILC2) and other lymphocytes to induce the production of Th2 cytokines. The cytokine IL-33 was also upregulated by radiation as recently suggested in cutaneous radiation injury. This gene product has been shown to attract ILC2 to specific tissue sites but may also stimulate other cells through its receptor ILIR1, which was constitutively expressed at high levels except in the 20-Gy positive control. Downregulation of GATA2, GATA3, and MYB, key genes that play a crucial role in hematopoiesis and immunity, was also observed. These data suggest that the marrow microenvironment following radiation favors the expression of genes involved not only in attracting ILC2 back to the marrow from peripheral sites, but also in stimulating them to release Th2 cytokines. Although the irradiated MEBM model of the microphysiological device does not contain peripheral tissues and thus cannot functionally recruit ILC2 to the marrow, the initial steps leading to establishment of Th2 responses can be examined nonetheless within the scope of a human response.

The bulk RNA sequencing described above was performed after a two-week period following proton irradiation, wherein samples were pooled by combining 3 samples from each radiation group into 1 sample, and 3 such samples (9 total chips) were acquired for each of the 4 groups. These samples were dissolved in Trizol, kept frozen at −80° C. until sequenced, and sequenced using 150 bp single end reads on an Illumina NovaSeq to a depth of approximately 40M reads per sample with poly-A depletion of rRNA. Once the samples had been sequenced and demultiplexed into FASTQ sample libraries, reads were mapped to the hg19 genome with the STAR aligner, and counts were obtained by using the quantMode GeneCounts flag in STAR. Differential expression was quantified with DESeq2. Experimental groups were clustered by principal component analysis after variance stabilizing transform, and plotted on a heatmap using the pheatmap ( ) R package to illustrate the top 20 differentially expressed genes ranked by variance in fold change between groups.

The proton treatments described above were performed at The Roberts Proton Therapy Center at the Perelman School of Medicine at the University of Pennsylvania. These resources were used to deliver targeted synchrotron proton radiation with pencil beam scanning by CT guidance to the MEBM models. MEBM model tissues that had been cultured for 3 weeks using establishment media and myelopoietic media in sequence (see Table 1) were irradiated at 0 Gy, 0.2 Gy, 2 Gy, and 20 Gy dosages using the proton beam scanning capabilities of the proton therapy center's accelerator. Specifically, the plate was placed in the plane orthogonal to the proton beam. In order to align the depth of the model with the penetration depth of the Bragg peak, a spread-out Bragg peak (SOBP) was created by summing individual Bragg peaks to create a cumulative depth dose profile.

As background, protons interact in matter with an atom or a nucleus thereof via several mechanisms which, primarily, are limited to Coulombic interactions with atomic electrons and nuclei, and nuclear interactions with atomic nuclei. Most interactions are Coulombic, which result in ionization of atoms, and loose electrons go on to ionize further in the vicinity of the atom from which they originated. Protons on average lose relatively little energy in individual ionizations and are not deflected as much. Protons will undergo hundreds of thousands of interactions per centimeter of material with each interaction removing energy from the primary particle. The frequency of energy loss events increases rapidly as the proton slows down before eventually losing all of the energy and coming to rest at a depth determined by the initial energy. As protons travel through media the linear energy transfer and the dose deposited rises sharply near the end of their range, creating a narrow peak in the dose distribution called a Bragg peak. Clinically useful proton systems must increase the width of the Bragg peak to adequately cover large target volumes. This is accomplished, as in the case of a proton pencil beam scanning system, by delivering successively shallower pristine Bragg peaks with decreasing weight to achieve a cumulative uniform dose over the entire depth of the target.

The clinical proton system undergoes a series of periodic quality and reliability checks at daily, monthly, and annual intervals to ensure accurate dose, depth range, beam properties, and imaging system performance. The proton pencil beam scanning system is capable of delivering arbitrary Bragg peak spot patterns in 3D volumes to reproduce user generated dose distributions. As it relates to the present disclosure, clinical treatment planning software was used to create dose patterns in volumetric CT images and the software generates the corresponding spot patterns that will be delivered by the pencil beam scanning system. A CT image of the 8×5 plate-based array of MEBM models was used to generate a spot pattern to deliver the appropriate doses to columns of the MEBM-based plate, with an empty column separating test columns. The computer-generated treatment plan was measured with a planar dose measuring device consisting of an array of ionization chambers and compared to the calculated dose pattern with excellent agreement.

v. Lung-Bone Marrow Multiorgan Model

The tremendous complexity of the hematopoietic system is best represented by the diverse functionality of mature blood cells produced by the bone marrow. Among the essential physiological functions of these cells is to provide the innate mechanisms of host defense against infectious agents and other foreign insults. During infection, host cells recognize invading pathogens and release soluble signals that activate the innate immune system, triggering a complex cascade of rapid, non-specific responses to increase the mobilization of phagocytic immune cells from the marrow and recruit them to the site of infection for clearance of pathogens.

To this end, the feasibility of reconstructing this complex physiological process of innate immunity using the MEBM model was explored. This work aimed to model innate immune responses to bacterial infection in the distal airways of the human lung, which represent one of the most common infections associated with a variety of clinical conditions. The second microphysiological device, described above as an airway-on-a-chip, was used for modeling a human small airway infection.

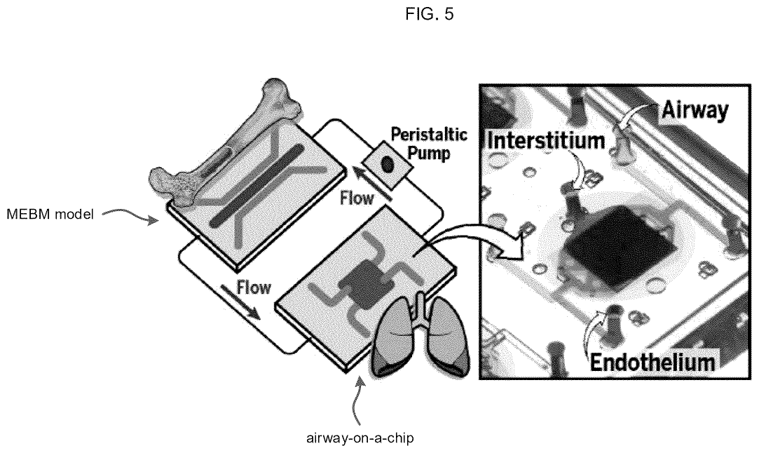

In order to evaluate the ability of the MEBM model of the microphysiological device, referred to here as the first microphysiological device, to react to infection elsewhere in the “body”, the second microphysiological device (i.e. airway-on-a-chip) must be connected to the first microphysiological device (i.e. bone marrow-on-a-chip), as is shown in FIG. 5. As in FIG. 5, the first microphysiological device and the second microphysiological device may be connected so as to construct a fluidically integrated system designed to emulate emergent multiorgan interactions along the lung-bone marrow axis during infection. The MEBM models were transitioned to circulation media (see Table 1) one day prior to airway infection experiments.

The airway model of the second microphysiological device, as described above, is a five-layer microfluidic device consisting of three culture chambers separated by two thin semipermeable membranes having 3 μm pores. In the second microphysiological device, primary human small airway epithelial cells were grown at the air-liquid interface (ALI) in the upper chamber, while type I collagen hydrogel containing primary human lung fibroblasts was formed in the middle chamber to reproduce the pulmonary interstitium (i.e. interstitium). The airway model of the second microphysiological device also included a monolayer of human pulmonary microvascular endothelial cells in the perfused lower chamber, mimicking the stromal-vascular interface in the multilayered airway tissue.

In an example, the MEBM model of the first microphysiological device and the airway model of the second microphysiological device are in a continuous fluidic circuit in which fluid flow is driven by a peristaltic pump at 10 μl/min. The peristaltic pump may be controlled by processing circuitry of a control module, in some examples, to generate a fluid flow profile suitable to a given application.

In an example, the side channels of the first microphysiological device are connected to the perfused lower chamber of the second microphysiological device.

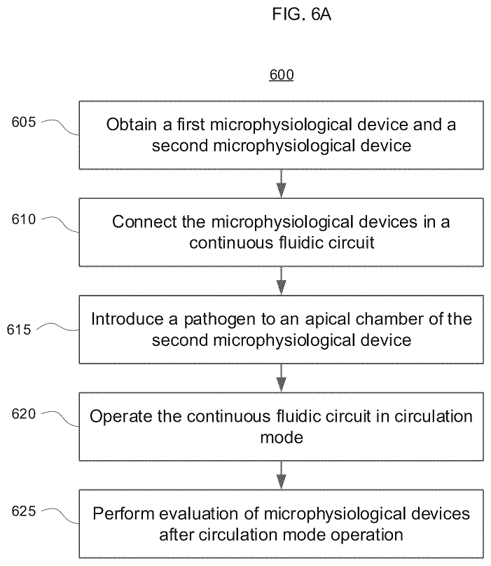

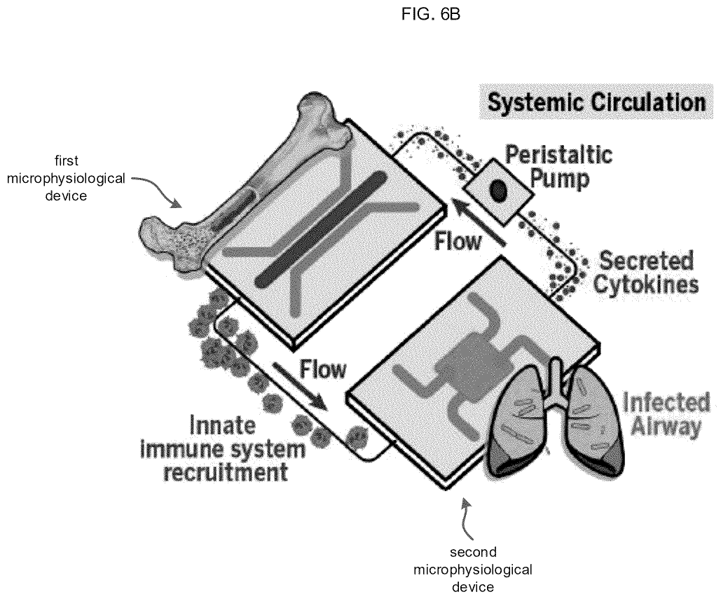

To this end, method 600 of FIG. 6A describes generating the multiorgan model and exposure of a first microphysiological device to soluble signals generated from infection of an airway model of a second microphysiological device.

At step 605 of method 600, a first microphysiological device and a second microphysiological device, fabricated as described above, are obtained.

At step 610 of method 600, the first microphysiological device and the second microphysiological device are connected in a continuous fluidic circuit. In an embodiment, a pump is arranged between the first microphysiological device and the second microphysiological device to control rate of fluid flow through the system. Air bubbles can be avoided during connection of tubing by ensuring that a fluid meniscus is always present at mating tubing-port junctions during tubing attachment. All manipulations of tubing and pumps should be performed quickly to avoid cooling of the incubator that housed the experiment.

At step 615 of method 600, a pathogen is introduced to the apical chamber of the second microphysiological device (housing the airway model).

In an example, Pseudomonas aeruginosa (P. aeruginosa) is used as a model pathogen that represents a common cause of airway infection. The GFP-expressing microbes are introduced into the airway compartment (i.e. apical chamber) to form a thin bacteria-laden film over the epithelial surface. Infection of the airway model using this method triggers rapid responses of the airway epithelium of the apical chamber, as has previously been demonstrated by a 27% increase in ciliary beat frequency as soon as 15 minutes following infection, approximating altered ciliary activity of infected native airways. SEM imaging of the epithelium after 2 days shows bacterial colonization of the airway microenvironment and its deleterious effects. Notably, the infected tissue is seen with localized regions of cellular denudation akin to epithelial damage of native human airways caused by Pseudomonas elastase-mediated disruption of tight junctions. It appears that the opening of these epithelial defects on the apical side provided an entry point for bacterial invasion into the epithelium. In addition to compromised epithelial integrity, bacterial infection in the airway model also led to significantly increased production of proinflammatory cytokines, including IL-8, CXCL5, IL-6, and G-CSF, which is important for the multiorgan model.

After infection of the airway model of the second microphysiological device, the continuous fluidic circuit of the multiorgan model is operated in circulation mode at step 620 of method 600. Step 620 of method 600 is performed for, for instance, 15 minutes, 30 minutes, 60 minutes, 90 minutes, 120 minutes, or 180 minutes, among others. The time during which circulation mode is permitted is based on the number of soluble signals generated by the infection of the airway model and the response, or lack thereof, by the MEBM model of the first microphysiological device.

In an example, following infection of the airway model apical surface with the procedure described in the previous sections, the system is permitted to operate in circulation mode for two hours. After this two-hour period, the microphysiological devices are carefully disconnected from the pump to avoid disruption by any sudden application of fluid flow, and evaluations are performed at step 625 of method 600.

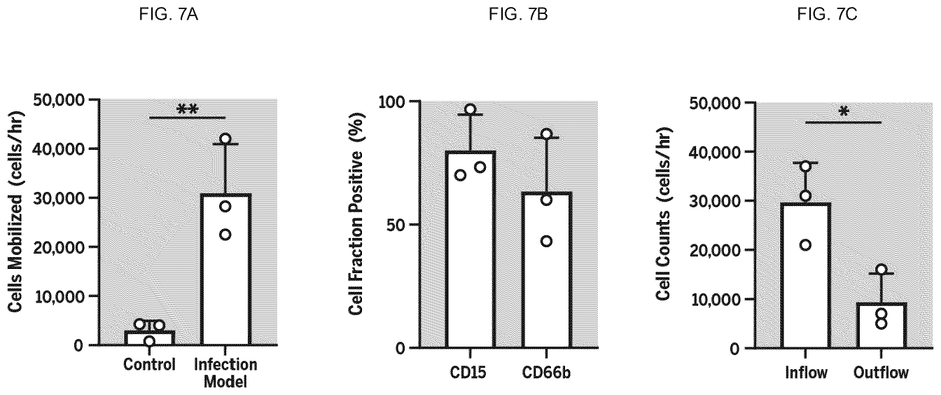

In experiments performed according to method 600, evaluations from step 625 of method 600 indicate, in one instance, that airway infection in the connected multiorgan system induced a 10-fold increase in cell mobilization from the first microphysiological device (i.e. the MEBM model) within 2 hours of bacterial introduction, presumably due to the elevated levels of inflammatory mediators in the recirculatory flow. Immunofluorescence analysis showed that 63% of the cells in circulation were mature neutrophils, approximating rapid and selective release of neutrophils from the bone marrow reserve during bacterial infection in vivo. We also found out that the number of neutrophils in the outflow from the second microphysiological device (i.e., the airway model) was significantly (p<0.02) lower than that entering the device, which indicated neutrophil recruitment and retention in the infected airway model. An illustration of the processes described above is shown in FIG. 6B. A graphical representation of the above-described processes is shown in FIG. 7A, FIG. 7B, and FIG. 7C. FIG. 7A shows a significant increase in the quantity of cells released from the MEBM model due to infection. FIG. 7B is a quantification of the type of mobilized cells, where CD15 and CD66b cells, both granulocyte markers, are observed in a majority of cells. FIG. 7C is a plot of the number of immune cells detected in the inflow and the outflow of the second microphysiological device.

For in situ examination of these recruited neutrophils, high-resolution imaging of the infected airway tissues was performed using SEM. Visualization of the vascular compartment revealed a large number of adherent neutrophils on the endothelial monolayer with various morphological characteristics that provided snapshots of the sequential steps of neutrophil recruitment during inflammatory responses. First, many of the imaged cells were in contact with the endothelial surface while retaining their round shape, similar to the morphology of leukocytes rolling on the endothelium. A subset of cells also displayed the phenotype of activated neutrophils as illustrated by their spreading and firm adhesion on the endothelial monolayer. Importantly, the images captured the dynamic process of neutrophil transmigration across the endothelium, during which extravasating neutrophils were identified by their cytoplasmic ruffling around the entry point on the luminal surface of the vasculature. The subsequent translocation of the cells into the subendothelial space was made visible by the formation of mound-like protrusions on the endothelial surface.

SEM imaging of the multiorgan model also made it possible to demonstrate and directly visualize the recruitment of neutrophils into the airspace of the second microphysiological device and the key features of their antimicrobial activities. Neutrophils in the interstitium were observed to migrate across the epithelial barrier and appear on the apical surface of the differentiated airway epithelium. Most of these cells exhibited a polarized morphology distinguished by lamellipodia- and uropod-like projections at opposite sides of the cell, which is characteristic of motile neutrophils undergoing directional migration. Of special note was the localized accumulation of neutrophils in areas of bacterial infection. These cells appeared to have direct interactions with Pseudomonas, but a large number were also seen with web-like fibrous structures emanating from the main cell body that were anchored to the epithelial surface and covered with small clusters of bacteria. The pericellular fibers were found throughout the infected tissue and shared the common feature of capturing bacteria, resembling extracellular traps released by neutrophils and other granulocytes to immobilize and destroy pathogens during innate immune responses. Indeed, these structures were stained positive for citrullinated histone H3, which is a key component of neutrophil extracellular traps (NETs). The recruited neutrophils also accumulated at sites of infection to form multicellular aggregates with NETs, most of which were entangled with large numbers of bacteria. This behavior replicates neutrophil swarming in vivo that represents a specialized host defense mechanism to enhance antimicrobial activities of neutrophils against large clusters of pathogens.

Although qualitative, this imaging data show the feasibility of using the multiorgan model to emulate neutrophil-mediated innate immunity in the integrated, organ-specific context of bacterial infection in the lung.

Regarding the airway bacterial infection and sampling described above, the airway was infected with GFP-expressing Pseudomonas aeruginosa (ATCC 15692GFP) that had been cultured in ATCC Medium 2854 (containing 300 μg/ml ampicillin) and diluted to a dose of approximately 20 bacteria per epithelial cell in the second microphysiological device. Thus, the Pseudomonas was infused into the airway devices at 20×106 bacteria/ml. Ciliary beat frequency was assayed by performing live imaging of the apical surface of the airway model and manually counting the interval in number of frames between cyclical beating of the cilia.

Effluent sampling for cytokine multiplexing was performed for the first time point immediately prior to the injection of bacteria. The bacteria were incubated within the apical airway channel of the second microphysiological device for two hours, during which period 25 μl of media was sampled from the vascular channel effluent at time points (0, 30, 60, and 120 minutes). Quantification of cytokine release from the devices was performed using a multiplexed Luminex assay panel for cytokine targets. Each single datapoint represents a pooled sample taken from 2 independent chips, and 4 such samples (from 8 chips total) were collected from each group (infected, non-infected) at each timepoint. Two chips per sample were used to attain the 50 μl sample volume required for each Luminex assay sample.

Statistically analyses described above were performed as follows. Mobilization of neutrophils in response to IL-8 and AMD3100 was assessed by ordinary one-way ANOVA, with Dunnett correction employed for multiple comparisons. Marker expression (CD15 and CD66b) as compared between AMD3100- and IL-8-mobilized groups was tested by unpaired t-test. Cell populations following irradiation were compared using 2-way ANOVA with multiple comparisons testing adjusted by Dunnett correction. Mobilization following infection was compared between infected and non-infected groups by unpaired t-test. Quantities of neutrophils at inflow and outflow position were compared using Welch's t-test to account for unequal variances in the data. Comparisons between multiplexed Luminex cytokine assays were performed using multiple t-tests, corrected by the Holm-Sidak method.

The present disclosure demonstrates an advanced engineering framework for assembling a multifunctional in vitro model of the hematopoietic vascular niche in the human marrow. The guiding principle of the MEBM model of the microphysiological device exploits the inherent properties of HSCs and vascular cells to self-organize an in vitro analog of the specialized human marrow microenvironment capable of essential hemopoietic function. This approach relies on similar principles to those required for the spontaneous generation of organoids and complex multicellular structures from self-organizing adult stem cells. This is unlike the conventional method of constructing organs-on-a-chip and microphysiological systems that often relies on predetermined selection and spatially defined patterning of required cell types.

Among the key demonstrations of this study is transcriptomic analysis of the MEBM model using scRNA-seq. This approach made it possible to interrogate and verify the biological complexity of the engineered hematopoietic niche. Data revealed that despite the initial simplicity, high levels of cellular heterogeneity emerged from the starting populations of HSCs, endothelial cells, fibroblasts, and mesenchymal stromal cells, which resulted in complex niche formation and multilineage differentiation (e.g., multilineage hematopoiesis). The high-dimensional single-cell analysis permitted identification and transcriptomic profiling of these cellular components. Of particular interest was the presence of non-hematopoietic stromal cells identified by specific markers of HSC-supporting bone marrow niche cells such as LEPR. Considering that such markers are not constitutively expressed in fibroblasts and mesenchymal stromal cells, this finding raises the possibility that these cells may have acquired the bone marrow-specific phenotype during in vitro development of the MEBM model. Based on extensive evidence demonstrating the reciprocal signaling between HSCs and niche cells, this may be understood as HSC-induced reprogramming of stromal cells in the starting population to increase their capacity to support HSCs and their hematopoietic function in the engineered microenvironment.

The scRNA-seq data also enabled advanced bioinformatics analysis of directional intercellular communication in the MEBM model. For each pair of different cell populations, this study yielded approximately 40-200 ligand-receptor interactions with statistically significant (p<0.01) cell type-specificity. These results demonstrated the presence of essential molecular hematopoietic signaling pathways including: KIT-KITLG, NOTCH-DLL, and CXCR4 CXCL12 reciprocal pairs, among many others. The data also revealed cell-cell interactions not previously described in the hematopoietic vascular niche of the marrow. For example, analysis of the endothelial cell-HSC pair showed the interaction of VEGF-A released by HSCs and progenitor cells with endothelial VEGFR-1. VEGF-A is an extensively studied angiogenic factor that also plays an indispensable role in hematopoiesis by acting in an autocrine fashion to promote the survival of HSCs. VEGF-A-mediated paracrine signaling between HSCs/progenitors and endothelial cells, as shown in the present disclosure, appears to be a new observation, which may suggest the potential role of hematopoietic cells in modulating vascular structure and function in the hematopoietic niche.

The cellular heterogeneity and ligand-receptor interactions discussed here represent the complexity of the MEBM model that spontaneously arises from the development of the engineered hematopoietic niche. Demonstration of neutrophil mobilization is another example of such complexity that occurs at higher levels of organization. In response to IL-8 in the vascular compartment, the MEBM model exhibited emergent behavior in which HSC-derived mature neutrophils became motile and migrated across the endothelium to intravasate and flow out of the construct, replicating the coordinated process of neutrophil egress from the bone marrow in vivo. Essential for modeling this complex physiological response was the self-assembled and externally accessible microvasculature distributed throughout the hydrogel scaffold. In addition to serving as the basis of the hematopoietic vascular niche, the integrated blood vessels provided a means to directly perfuse the marrow construct in a controlled manner. This is an important feature of the MEBM model that makes it possible to mimic the dynamic process of cellular trafficking between the hematopoietic niche and blood flow.

By leveraging this capability, the proof-of-principle of engineering a human lung-bone marrow multiorgan system with circulatory flow of common media to model the physiological function of neutrophils as the first responders and primary effector cells of the innate immune system was demonstrated. In response to Pseudomonas infection in the airspace of the airway model (i.e. second microphysiological device), this integrated model reproduced the entire cascade of neutrophil recruitment from bone marrow mobilization to diapedesis to bacterial phagocytosis and NETosis in a completely spontaneous manner. These data provide qualitative evidence that the multiorgan model can recapitulate key aspects of early innate immune responses at the whole-body level—that is, at the level of multiple interacting organs. This level of physiological realism has not been demonstrated in existing in vitro models of the human marrow, highlighting the potential of the models of the present disclosure as enabling platforms for modeling human immunity in vitro.

The bone marrow-on-a-chip technology of the present disclosure represents a significant advance in the current state-of-the-art for in vitro studies of, at least, human hematopoiesis and immunity. The biologically inspired design principle and array-based scalable instrumentation of the MEBM model may enable the development of new approaches that permit both biological complexity and experimental practicality in modeling the human hematopoietic system and its physiological function. As shown by the radiotherapy-induced hematoablation model, the MEBM model also provides a platform to create other specialized in vitro models with direct relevance to current clinical practice, which may facilitate the development of new therapeutics and treatment strategies. Further, the demonstration of the interconnected lung-marrow model makes possible the use of the bone marrow-on-a-chip as an immediately deployable immune organ module in multiorgan systems for simulating immune responses in the integrated context of the human body.

Lastly, the present disclosure represents an important step forward in leveraging advanced bioanalytical techniques, such as scRNA-seq, to explore previously inaccessible dimensions of complexity in engineered tissue models. When combined with scalable experimental platforms, this approach may provide new opportunities to generate large high-content data sets from complex, entirely human cell-based in vitro models. As demonstrated above, these types of data can be used to reveal and probe the depth of biology for validating the physiological relevance of the model but may also inform the process of model development to create more realistic and predictive in vitro analogs of human physiological systems. While much developmental work remains, there is great potential in harnessing the power of these micro-engineered tools to tackle the long-standing challenge of using cultured cells to reverse engineer the complexity of human hematopoiesis and immunity.

Obviously, numerous modifications and variations are possible considering the above teachings. It is therefore to be understood that within the scope of the appended claims, embodiments of the present disclosure may be practiced otherwise than as specifically described herein.

Embodiments of the present disclosure may also be as set forth in the following parentheticals.

(1) A microphysiological device, comprising two or more side channels, each of the two or more side channels having endothelial cells therein, and at least one central channel arranged therebetween, the at least one central channel having a cellularized scaffold formed therein, the cellularized scaffold of the at least one central channel including hematopoietic stem cells, mesenchymal stromal cells, and endothelial cells.

(2) The microphysiological device of 1, further comprising vasculature developed within and between the two or more side channels and the at least one central channel.

(3) The microphysiological device of either one of (1) or (2), wherein the vasculature developed within and between the two or more side channels and the at least one central channel includes anastomoses formed between vessels within the at least one central channel and an endothelium formed within the two or more side channels, the anastomoses permitting perfusion between the two or more side channels.

(4) The microphysiological device of any one of (1) to (3), wherein the cellularized scaffold of the at least one central channel includes myeloid cells.

(5) The microphysiological device of (4), wherein the myeloid cells are differentiated from a portion of the hematopoietic stem cells within the cellularized scaffold of the at least one central channel.

(6) The microphysiological device of either one of (4) or (5), wherein the myeloid cells include macrophages and erythroid cells.

(7) The microphysiological device of any one of (4) to (6), wherein the myeloid cells include neutrophils that mobilize from the cellularized scaffold of the at least one central channel in response to exposure to challenging cytokines.

(8) The microphysiological device of either one of (6) or (7), wherein the macrophages and erythroid cells are erythropoietin induced.

(9) The microphysiological device of any one of (1) to (8), wherein the hematopoietic stem cells are human hematopoietic stem cells.

(10) The microphysiological device of any one of (1) to (9), wherein the cellularized scaffold includes an extracellular matrix (ECM) within which the hematopoietic stem cells, the mesenchymal stromal cells, and the endothelial cells are seeded, a number of human hematopoietic stem cells within the ECM after vascularization being at least as many as a number of human hematopoietic stem cells within the ECM when seeded.

(11) The microphysiological device of any one of (1) to (10), wherein the cellularized scaffold includes one or more of polylactic acid, polyglycolic acid, poly-lactic-co-glycolic acid, polycaprolactone, fibrin, fibrinogen, extracellular matrix, collagen, chitosan, proteoglycans, gelatin, and agarose.

(12) The microphysiological device of any one of (1) to (11), wherein the hematopoietic stem cells are differentiated into multiple cell lineages.

(13) The microphysiological device of any one of (1) to (12), wherein the hematopoietic stem cells are differentiated into myeloid progenitor cells.

(14) The microphysiological device of (13), wherein, wherein the cellularized scaffold of the at least one central channel includes myeloid cells and the myeloid cells are differentiated from a portion of the myeloid progenitor cells.

(15) The microphysiological device of any one of (1) to (14), wherein the hematopoietic stem cells are differentiated into lymphoid progenitor cells.

(16) A microphysiological system for multi-organ modeling, comprising a first microphysiological device having two or more side channels and at least one central channel arranged therebetween, each of the two or more side channels having endothelial cells therein, the at least one central channel having a cellularized scaffold formed therein, the cellularized scaffold of the at least one central channel including hematopoietic stem cells, mesenchymal stromal cells, and endothelial cells, and wherein the first microphysiological device includes vasculature developed within and between the two or more side channels and the at least one central channel, a second microphysiological device having a plurality of chambers, the plurality of chambers including an apical chamber having epithelial cells therein, a central chamber having a cellularized scaffold formed therein, the cellularized scaffold of the central chamber including fibroblasts, and a basal chamber having endothelial cells therein, and a pump arranged in a fluidic circuit with the first microphysiological device and the second microphysiological device, an outflow of the first microphysiological device being an inflow to the second microphysiological device, an outflow of the second microphysiological device being an inflow to the first microphysiological device.

(17) The microphysiological system according to (16), wherein the outflow of the first microphysiological device is provided by a first one of the two or more side channels of the first microphysiological device and the inflow to the second microphysiological device is provided to the apical chamber of the second microphysiological device.

(18) The microphysiological system according to either one of (16) or (17), wherein the outflow of the second microphysiological device is provided by the basal chamber of the second microphysiological device and the inflow to the first microphysiological device is provided to a second one of the two or more side channels of the first microphysiological device.

(19) The microphysiological system of any one of (16) to (18), further comprising vasculature developed within and between the two or more side channels and the at least one central channel of the first microphysiological device.

(20) The microphysiological system of any one of (16) to (19), wherein the vasculature developed within and between the two or more side channels and the at least one central channel of the first microphysiological device includes anastomoses formed between vessels within the at least one central channel and an endothelium formed within the two or more side channels, the anastomoses permitting perfusion between the two or more side channels.

(21) The microphysiological system of any one of (16) to (20), wherein the cellularized scaffold of the at least one central channel of the first microphysiological device includes myeloid cells.

(22) The microphysiological system of (21), wherein the myeloid cells are differentiated from a portion of the hematopoietic stem cells within the cellularized scaffold of the at least one central channel of the first microphysiological device.

(23) The microphysiological system of either one of (21) or (22), wherein the myeloid cells include macrophages and erythroid cells.

(24) The microphysiological system of any one of (21) to (23), wherein the myeloid cells include neutrophils that mobilize from the cellularized scaffold of the at least one central channel of the first microphysiological device in response to exposure of the apical chamber of the second microphysiological device to challenging cytokines.

(25) The microphysiological system of either one of (23) or (24), wherein the macrophages and erythroid cells are erythropoietin induced.

(26) The microphysiological system of any one of (16) to (25), wherein the hematopoietic stem cells are human hematopoietic stem cells.

(27) The microphysiological system of any one of (16) to (26), wherein the cellularized scaffold of the first microphysiological device includes an extracellular matrix (ECM) within which the hematopoietic stem cells, the mesenchymal stromal cells, and the endothelial cells are seeded, a number of human hematopoietic stem cells within the ECM after vascularization being at least as many as a number of human hematopoietic stem cells within the ECM when seeded.

(28) The microphysiological system of any one of (16) to (27), wherein the cellularized scaffold of the first microphysiological device includes one or more of polylactic acid, polyglycolic acid, poly-lactic-co-glycolic acid, polycaprolactone, fibrin, fibrinogen, extracellular matrix, collagen, chitosan, proteoglycans, gelatin, and agarose.

(29) The microphysiological device of any one of (16) to (28), wherein the hematopoietic stem cells are differentiated into multiple cell lineages.

(30) The microphysiological device of any one of (16) to (29), wherein the hematopoietic stem cells are differentiated into myeloid progenitor cells.

(31) The microphysiological device of (30), wherein the cellularized scaffold of the at least one central channel includes myeloid cells, and wherein the myeloid cells are differentiated from a portion of the myeloid progenitor cells.

(32) The microphysiological device of any one of (16) to (31), wherein the hematopoietic stem cells are differentiated into lymphoid progenitor cells.

(33) A method of preparing a microphysiological device comprising two or more side channels and at least one central channel arranged therebetween, comprising: a) contacting a scaffold of the at least one central channel with a plurality of hematopoietic stem cells, mesenchymal stromal cells, and endothelial cells, forming a cellularized scaffold in the at least one central channel, and b) contacting each of the two or more side channels with a plurality of endothelial cells.

(34) The method of (33), further comprising developing vasculature within and between the two or more side channels and the at least one central channel.

(35) The method of either one of (33) or (34), wherein the vasculature developed within and between the two or more side channels and the at least one central channel includes anastomoses formed between vessels within the at least one central channel and an endothelium formed within the two or more side channels, the anastomoses permitting perfusion between the two or more side channels.

(36) The method of any one of (33) to (35), wherein the cellularized scaffold of the at least one central channel includes myeloid cells.

(37) The method of (36), wherein the myeloid cells are differentiated from a portion of the hematopoietic stem cells within the cellularized scaffold of the at least one central channel.

(38) The method of either one of (36) or (37), wherein the myeloid cells include macrophages and erythroid cells.

(39) The method of any one of (36) to (38), wherein the myeloid cells include neutrophils that mobilize from the cellularized scaffold of the at least one central channel in response to exposure to challenging cytokines.

(40) The method of any one of (36) to (39), wherein the macrophages and erythroid cells are erythropoietin induced.

(41) The method of any one of (33) to (40), wherein the hematopoietic stem cells are human hematopoietic stem cells.

(42) The method of any one of (33) to (41), wherein the cellularized scaffold includes an extracellular matrix (ECM) within which the hematopoietic stem cells, the mesenchymal stromal cells, and the endothelial cells are seeded, a number of human hematopoietic stem cells within the ECM after vascularization being at least as many as a number of human hematopoietic stem cells within the ECM when seeded.

(43) The method of any one of (33) to (42), wherein the cellularized scaffold includes one or more of polylactic acid, polyglycolic acid, poly-lactic-co-glycolic acid, polycaprolactone, fibrin, fibrinogen, extracellular matrix, collagen, chitosan, proteoglycans, gelatin, and agarose.

(44) The method of any one of (33) to (43), wherein the hematopoietic stem cells are differentiated into multiple cell lineages.

(45) The method of any one of (33) to (44), wherein the hematopoietic stem cells are differentiated into myeloid progenitor cells.

(46) The method of (45), wherein the cellularized scaffold of the at least one central channel includes myeloid cells, and wherein the myeloid cells are differentiated from a portion of the myeloid progenitor cells.

(47) The method of any one of (33) to (46), wherein the hematopoietic stem cells are differentiated into lymphoid progenitor cells.

Thus, the foregoing discussion discloses and describes merely exemplary embodiments of the present disclosure. As will be understood by those skilled in the art, the present disclosure may be embodied in other specific forms without departing from the spirit thereof. Accordingly, the disclosure of the present disclosure is intended to be illustrative, but not limiting of the scope of the disclosure, as well as other claims. The disclosure, including any readily discernible variants of the teachings herein, defines, in part, the scope of the foregoing claim terminology such that no inventive subject matter is dedicated to the public.

The invention claimed is:

1. A microphysiological device, comprising:
two or more side channels, each of the two or more side channels having endothelial cells therein, and
at least one central channel arranged therebetween, the at least one central channel having a cellularized scaffold formed therein, the cellularized scaffold of the at least one central channel including hematopoietic stem cells, mesenchymal stromal cells, myeloid cells, and endothelial cells.

2. The microphysiological device of claim 1, further comprising vasculature developed within and between the two or more side channels and the at least one central channel.

3. The microphysiological device of claim 2, wherein the vasculature developed within and between the two or more side channels and the at least one central channel includes anastomoses formed between vessels within the at least one central channel and an endothelium formed within the two or more side channels, the anastomoses permitting perfusion between the two or more side channels.

4. (canceled)

5. The microphysiological device of claim 1, wherein the myeloid cells are differentiated from a portion of the hematopoietic stem cells within the cellularized scaffold of the at least one central channel.

6. The microphysiological device of claim 1, wherein the myeloid cells include macrophages and erythroid cells.

7. The microphysiological device of claim 1, wherein the myeloid cells include neutrophils that mobilize from the cellularized scaffold of the at least one central channel in response to exposure to challenging cytokines.

8. The microphysiological device of claim 6, wherein the macrophages and erythroid cells are erythropoietin induced.

9. The microphysiological device of claim 1, wherein the hematopoietic stem cells are human hematopoietic stem cells.

10. The microphysiological device of claim 2, wherein the cellularized scaffold includes an extracellular matrix (ECM) within which the hematopoietic stem cells, the mesenchymal stromal cells, myeloid cells, and the endothelial cells are seeded, a number of human hematopoietic stem cells within the ECM after vascularization being at least as many as a number of human hematopoietic stem cells within the ECM when seeded.

11. The microphysiological device of claim 1, wherein the cellularized scaffold includes one or more of polylactic acid, polyglycolic acid, poly-lactic-co-glycolic acid, polycaprolactone, fibrin, fibrinogen, extracellular matrix, collagen, chitosan, proteoglycans, gelatin, and agarose.

12. The microphysiological device of claim 1, wherein the hematopoietic stem cells are differentiated into multiple cell lineages.

13. The microphysiological device of claim 1, wherein the hematopoietic stem cells are differentiated into myeloid progenitor cells.

14. The microphysiological device of claim 13, wherein the cellularized scaffold of the at least one central channel includes myeloid cells, and wherein the myeloid cells are differentiated from a portion of the myeloid progenitor cells.

15. The microphysiological device of claim 1, wherein the hematopoietic stem cells are differentiated into lymphoid progenitor cells.

16. A microphysiological system for multi-organ modeling, comprising:
a first microphysiological device having two or more side channels and at least one central channel arranged therebetween,
each of the two or more side channels having endothelial cells therein, the
at least one central channel having a cellularized scaffold formed therein, the cellularized scaffold of the at least one central channel including hematopoietic stem cells, myeloid cells, mesenchymal stromal cells, and endothelial cells, and
wherein the first microphysiological device includes vasculature developed within and between the two or more side channels and the at least one central channel;
a second microphysiological device having a plurality of chambers, the plurality of chambers including
an apical chamber having epithelial cells therein,
a central chamber having a cellularized scaffold formed therein, the cellularized scaffold of the central chamber including fibroblasts, and
a basal chamber having endothelial cells therein; and
a pump arranged in a fluidic circuit with the first microphysiological device and the second microphysiological device, an outflow of the first microphysiological device being an inflow to the second microphysiological device, an outflow of the second microphysiological device being an inflow to the first microphysiological device.

17-32. (canceled)

33. A method of preparing a microphysiological device comprising two or more side channels and at least one central channel arranged therebetween, comprising:
a) contacting a scaffold of the at least one central channel with a plurality of hematopoietic stem cells, mesenchymal stromal cells, myeloid cells, and endothelial cells, forming a cellularized scaffold in the at least one central channel; and
b) contacting each of the two or more side channels with a plurality of endothelial cells.

34-47. (canceled)
